SEQUENCE LISTING (10 sequences)

SEQ ID NO: 1 - DNA, 3687 bp, Mus musculus
tcagcagggacaacgcccgcaggctaggcatgaaggcggcgacaccggtggtggtgacggcggctgctcctgcgatggagccgggcccttctgtgtccccggggccttcgcgctccttcaaagaggagctgctgtgtgccgtgtgctacgacccgttccgcgacgcagtaactctgcgctgtggccacaacttctgccgccggtgcgtgagcggctgctgggaggtgcagacgacgccctcgtgtccggtgtgcaaggaacgagcggtgcccggggagctgcgcaccaaccacacgctcaacaacctggtggagaccttgctgcgcgaggaggctgagggcgcgcgctggaccggtcgccggtccccgcgcccctgccgtgcgcaccgtgccccgctcacgctcttctgcctggaggacaaggagctgctgtgctgtgcttgccaggccgacgcccggcaccaagagcatcgtgtgcagcccatcaaggacactgcgcaagacttccgggccaagtgtaagaacatggagcatgtattgcgagagaaagccaaggccttctgggccctgaggcgcacctatgaggccattgccaagcacaatgaggtgcaaacgacttggctggaaggccgcatccgggatgagtttgacaagctccgtgatttcctgagggtggaggaacaggccaccttggatgccatgaaggaagagagcagaaagaagcacctgcaggctgaggagaagatgaagcagctagcagaacagaccgaggcgctggctcgggagattgagcgtctgcagatggagatgaaggaagatgacatgaccttcctcatgaaacacaagagccgaaaacgccggctcttctgcaccgtggagccagctcctctccagcctggcttgctaatggatgcatgcaagtatctggagtccctgcagtaccgagtctggaagaagatgcttggatccgttgagtctgtgcccttcagcttggatcccaacacagctgctggctggctcaaagtggctgatgacctcacgagtgtcatcaaccatggctaccgcgtgcaagtggagaatccagagcgcttctcctcggcaccctgcctgctaggctctcaagtgttctccaagggctcccactcttgggaggtggatgtgggtggcctgccaacctggcgagtgggtgtggttcgggtgcaggcacatgcacaggcgcaggctcaggctgacgtaggtggtgaaggccactcacacagctgctaccatgatacacgctcaggcttctggtacctgtgccgcacgcagggtgtggatggagaccactgcatgacttccgacactgccacagcccctctggtccaggccatgccgcgccgtctgcgtgtggagctggagtgtgaggagggtgagctatccttctatgactctgagcgccactgccatctgtataccttccatgcccactttggggaggtgaggccctacttctacctgggagcctctcgaggtgacggtcccccggaacctctgcgtatctgccacctgcgtgtctccatcaaagaagagctggacatctgagctgcccacccctgacacatgcagcatactatatcctgtcttagcttttctgtagctccaaagttaggagccacccaggaggtgcttggctgagcctaggctctgtctacagtcatgctaccttcaggatgtgggttttctgttcttggattgctggtatactgttttctttgtaggatggcattataatgtaggtgtagactatttttagagatgatgaagccagcctatcaggagatgcctcttattgagtctatttgtcatttatgtttcccaggaagaggtccttgtcaggccacacagggaagccccaggatggtttgagacaagagtgggcaaagccttcacctaggctctcccaccccagtaggacaagttaggtattggccagcctcactgagctatgcatggctttagcatctggtctgaaccaggagtccccgtgttggttcctaggacaggattgtccttgactctctctgtgggaacactgtagggtgtgaggtactctggaatacagctcagagttgtgggtgtctcaggaaaggcagctccagggcctttggcagtggttaaggactgaccagcctcaagttagtgccatagaggccaaagcaccagaatgcagtgagtgagaagacagctagtgctgggttggaacatgagccccactctgttactcagtctttcggcacggaatcgacactggttggtcacctcatgctttgaacgtttcctctggaattgtccagttttctagaacactttttaaacctgtgtttccacatctgtgatttgacactagtccttggaaatcactggaggaaggtatgagaaggagcctcagagaatgctgcttcagtcagtgactgttggtctgggagacgagtcctggccttgctggccgttgtcttcattggcttccacctgcctgtccttgctctgctctctgcagattccacccctttcctttatgtgtccctctgccttttctttctcagtcatgcctgtagatggagtctaattgccaactaaccaaatcccaagagatgttgtaaggaaaaaataccatctcaggggtgccctgtcatcgtcaagacctaggacctagcatcccaatttcagcctgcacccctcattacataagacttgttttaaaccacgccgattacccactaattggctctaaatgggtcatgtgcacttgtggattatctaacaagtggagacacagaagaaaccctggtgcaggccaggccgggagcagggacagtgttggcaagccagcttgtgagtgtcagatgcttgggcaccacggatgtgaaaggtgcgcctggtgcaatgtatgtgtttggttaaagaaactctctgaaattactgttataataagtttttaaaagttttttctttctttttaattttcaccttaactcttaaatagggtaatttcaatgacctagactcttagaaaaaatttgacttaccccacaactgacatgtttctttcagagcttttgtaaacacaaattcctagtgtaacttgtacctgatctgtccttcccattgtaagattccatcgtgtgcagtgaatgtgctgtgcaacttgttagttgatggacatttggcttgttgcagtgtgtttagctgttatgagttctgctgctatgaaatttgtgtgcaagttttgtgtggctgtgtctcttgattttctgtcatataccttggagtctgttttccaggtcatatgataattattttaagccctttggggacttgccaagctgtttctaagagagccgtcccattttctactcccaccggcagcaggtgagggctggtttgtaacgaattctctctgccctcttaagctgaggaagctggagtaggtctcatttgccctgtagttgcgatctctgatggctggggagcatctttcctcatgtttgctgtgtatctgcttcagagacttcagggtgtttgcccattgggttgtctgaccttttattatgaaggtttacaagtttgttatgcattctagataaaagttcctttgtgtcagatgaatcacataaaaattttcctcctaattcaaaaaaaaaaaaaaaaa

SEQ ID NO: 2 - protein, 501 aa, Mus musculus
MEPGPSVSPGPSRSFKEELLCAVCYDPFRDAVTLRCGHNFCRRCVSGCWEVQTTPSCPVCKERAVPGELRTNHTLNNLVETLLREEAEGARWTGRRSPRPCRAHRAPLTLFCLEDKELLCCACQADARHQEHRVQPIKDTAQDFRAKCKNMEHVLREKAKAFWALRRTYEAIAKHNEVQTTWLEGRIRDEFDKLRDFLRVEEQATLDAMKEESRKKHLQAEEKMKQLAEQTEALAREIERLQMEMKEDDMTFLMKHKSRKRRLFCTVEPAPLQPGLLMDACKYLESLQYRVWKKMLGSVESVPFSLDPNTAAGWLKVADDLTSVINHGYRVQVENPERFSSAPCLLGSQVFSKGSHSWEVDVGGLPTWRVGVVRVQAHAQAQAQADVGGEGHSHSCYHDTRSGFWYLCRTQGVDGDHCMTSDTATAPLVQAMPRRLRVELECEEGELSFYDSERHCHLYTFHAHFGEVRPYFYLGASRGDGPPEPLRICHLRVSIKEELDI

SEQ ID NO: 3 - DNA, 4196 bp, Homo sapiens
ctcgggtcgcgggcgttggcagccgggcgggtgggaggggccggagcaaaagttccgggcgcccgagccggctgctcgtgccatggagcggagtcccgacgtgtcccccgggccttcccgctccttcaaggaggagttgctctgcgccgtctgctacgaccccttccgcgacgcagtcactctgcgctgcggccacaacttctgccgcgggtgcgtgagccgctgctgggaggtgcaggtgtcgcccacctgcccagtgtgcaaagaccgcgcgtcacccgccgacctgcgcaccaaccacaccctcaacaacctggtggagaagctgctgcgcgaggaggccgagggcgcgcgctggaccagctaccgcttctcgcgtgtctgccgcctgcaccgcggacagctcagcctcttctgcctcgaggacaaggagctgctgtgctgctcctgccaggccgacccccgacaccaggggcaccgcgtgcagccggtgaaggacactgcccacgactttcgggccaagtgcaggaacatggagcatgcactgcgggagaaggccaaggccttctgggccatgcggcgctcctatgaggccatcgccaagcacaatcaggtggaggctgcatggctggaaggccggatccggcaggagtttgataagcttcgcgagttcttgagagtggaggagcaggccattctggatgccatggccgaggagacaaggcagaagcaacttctggccgacgagaagatgaagcagctcacagaggagacggaggtgctggcacatgagatcgagcggctgcagatggagatgaaggaggacgacgtttcttttctcatgaaacacaagagccgaaaacgccgactcttctgcaccatggagccagagccagtccagcccggcatgcttatcgatgtctgcaagtacctgggctccctgcagtaccgcgtctggaagaagatgcttgcatctgtggaatctgtacccttcagctttgaccccaacaccgcagctggctggctctccgtgtctgacgacctcaccagcgtcaccaaccatggctaccgcgtgcaggtggagaacccggaacgcttctcctcggcgccctgcctgctgggctcccgtgtcttctcacagggctcgcacgcctgggaggtggcccttggggggctgcagagctggagggtgggcgtggtacgtgtgcgccaggactcgggcgctgagggccactcacacagctgctaccacgacacacgctcgggcttctggtatgtctgccgcacgcagggcgtggagggggaccactgcgtgacctcggacccagccacgtcgcccctggtcctggccatcccacgccgcctgcgtgtggagctggagtgtgaggagggcgagctgtctttctatgacgcggagcgccactgccacctgtacaccttccacgcccgctttggggaggttcgcccctacttctacctggggggtgcacggggcgccgggcctccagagcctttgcgcatctgccccttgcacatcagtgtcaaggaagaactggatggctgagctggcccggggctgccccggtcttgtgccacagcactgttttctttctgccctcttcctaatgcccacactgcttgggcactattgcgcccctgcctccttgccaggctcttcctcctgtcctgcctggtccttttccatgactccaggctgtgcctctctccatgtttggtcccttctgtgcccatggtcaggagctattcgggtggcacctcgctggccaggctctcccgagtcgtggcacctccacaatgtgaattttctgaatccctattccaggatttctgggaataatgtttacttctagaatgggcctgttgtaaaccatctcatcgaggtgtggtaaagccattggatgaggaggggactgccatggaaaggagagtttgttacttacggttctgagaggaggggccacataggaaagccccacggtgggtcagaaggcggaaggagggaggggaacgtgtgggcaagagacttcctctggtttcctcaggaggaaatgggcaaggcagagtaagcaggggagacaggtttaagggtagctggcttgagtaatttcagtggctctcaggataggggctgccctttttgtctgatacctggccccgggatagtcaggacaggtgaatgttggcctggggtgtgacagccctgggagagccatgtgaaggaggcagctggcgccatcgctccggattagttggtttccataggaaaggcatgctttcagccagatgcttgccatctctagggattgggggattggctagcctgggaggatcagtctgtccaggtcagcgaggccccagataccagagcatcaagagtacaggaaatacagttaatgcagggcctctgtgtggctggatcctccgtctccatcagatcagctctgattgatctattcttgcacgatttcctctgaacacagggttccagagtacttaaacacaacattttttaaatcgtgatttcggcctatttccttgccaggcctgtttccccaccaggaaatgagataggaggactggatgaggatgtcctgttatagttgctgtggaggaagttcctctggttaattctcatcagcgtctgcagaaaagaaggaaagagggcacccttttcagttgggaagaaaggagaggggtggcgccatggacgtggccctaaacgctgtgggagagggaagaggaggctgggcctcgctgccctcttgtctctgctgacttcagcctggtcatgcttgctctgccacttgcgatttcatccctaatttcttcctccaccatgcctgcagacttttccctgggcttgttttttctcgcacatctctgaagagtttttaatcttcagctcatcatgtcccaggaagtggcatcataaaaggaaatatttttttttcctaggagcagtgttaaaatctgggtcacattcctgaccaaggacagcatcctgccttctgcccatccccttcagttcacaaaagctgacattttaaacaaatcatgactcacacgtattaattggttataaatatgttgtgtacactggttagataaaacttaaggccacaaggagggcccaggtaggcgatgtcagtgtgtgaaggggctggattgggcgtggtgaggatgttggcaaaccagtgcatgcacctggttggaagatgctcagcctcacaaaagctccaagccctttgggagccaaagtgtctgagagtgtgaccctctcctgtaaagtatttatcccacccattaatataatttctgtataataaacttgacctgaaattatttcattctttatattaaacttttaaaaatgttttttattttcaccttagatatgggaagagttttttttttttttttttttttttaacaggataacttgagcaggctaggcctcttaaaaaaaaatttgagctaaaactcatttttcttttggcattttcttttcaatgttcttataagcaaagttcatccatgttgtagcatgtgttcaactttattttttcatcgggtaatattccattgtatggaatggtagtactacattttatttatcatgcatcgattggtggacatttggatcgtttctacttcttgactattatacataatgctgctaggaacttttgtgtatgagtttttgtgtggacatatgttttcatttctctttggtatatgcctgggagcagatttgctggatcatatgaaactctatttaacccttgagggactcccaaactgttttccaatgtggtgacacaattttatatcccatcaacagggcatgagggttctgatgactccacatccctcagtgctttttattatctatcttttaacttagccatcctagtaggggtaatgtggcatctcattgtgattttggtttgcatttccctgatggcgaatgatattgagcatcttttcatgagcttattggccatttgcatatcttcttaggacagctatctttagatcacttgctcattttttaattgggttatttgtctttttattattgagttgtaagagtccttttatagcctggcacaagtccctttaactggtatatgattataaaatttttctccgtgagctgtttcatttccttgatg

SEQ ID NO: 4 - protein, 493 aa, Homo sapiens
MERSPDVSPGPSRSFKEELLCAVCYDPFRDAVTLRCGHNFCRGCVSRCWEVQVSPTCPVCKDRASPADLRTNHTLNNLVEKLLREEAEGARWTSYRFSRVCRLHRGQLSLFCLEDKELLCCSCQADPRHQGHRVQPVKDTAHDFRAKCRNMEHALREKAKAFWAMRRSYEAIAKHNQVEAAWLEGRIRQEFDKLREFLRVEEQAILDAMAEETRQKQLLADEKMKQLTEETEVLAHEIERLQMEMKEDDVSFLMKHKSRKRRLFCTMEPEPVQPGMLIDVCKYLGSLQYRVWKKMLASVESVPFSFDPNTAAGWLSVSDDLTSVTNHGYRVQVENPERFSSAPCLLGSRVFSQGSHAWEVALGGLQSWRVGVVRVRQDSGAEGHSHSCYHDTRSGFWYVCRTQGVEGDHCVTSDPATSPLVLAIPRRLRVELECEEGELSFYDAERHCHLYTFHARFGEVRPYFYLGGARGAGPPEPLRICPLHISVKEELDG

SEQ ID NO: 5 - DNA, 4339 bp, Homo sapiens
ctcgggtcgcgggcgttggcagccgggcgggtgggaggggccggagcaaaagttccgggcgcccgagccggctgctcgtgccatggagcggagtcccgacgtgtcccccgggccttcccgctccttcaaggaggagttgctctgcgccgtctgctacgaccccttccgcgacgcagtcactctgcgctgcggccacaacttctgccgcgggtgcgtgagccgctgctgggaggtgcaggtgtcgcccacctgcccagtgtgcaaagaccgcgcgtcacccgccgacctgcgcaccaaccacaccctcaacaacctggtggagaagctgctgcgcgaggaggccgagggcgcgcgctggaccagctaccgcttctcgcgtgtctgccgcctgcaccgcggacagctcagcctcttctgcctcgaggacaaggagctgctgtgctgctcctgccaggccgacccccgacaccaggggcaccgcgtgcagccggtgaaggacactgcccacgactttcgggtgaggagcctgatagccgaagagagaaggaactttttaccaactcaccagtggatagtgacaaagacaaggctccaaaccagctctcctaaccttcagtccaggaggcaaggccaagtgcaggaacatggagcatgcactgcgggagaaggccaaggccttctgggccatgcggcgctcctatgaggccatcgccaagcacaatcaggtggaggctgcatggctggaaggccggatccggcaggagtttgataagcttcgcgagttcttgagagtggaggagcaggccattctggatgccatggccgaggagacaaggcagaagcaacttctggccgacgagaagatgaagcagctcacagaggagacggaggtgctggcacatgagatcgagcggctgcagatggagatgaaggaggacgacgtttcttttctcatgaaacacaagagccgaaaacgccgactcttctgcaccatggagccagagccagtccagcccggcatgcttatcgatgtctgcaagtacctgggctccctgcagtaccgcgtctggaagaagatgcttgcatctgtggaatctgtacccttcagctttgaccccaacaccgcagctggctggctctccgtgtctgacgacctcaccagcgtcaccaaccatggctaccgcgtgcaggtggagaacccggaacgcttctcctcggcgccctgcctgctgggctcccgtgtcttctcacagggctcgcacgcctgggaggtggcccttggggggctgcagagctggagggtgggcgtggtacgtgtgcgccaggactcgggcgctgagggccactcacacagctgctaccacgacacacgctcgggcttctggtatgtctgccgcacgcagggcgtggagggggaccactgcgtgacctcggacccagccacgtcgcccctggtcctggccatcccacgccgcctgcgtgtggagctggagtgtgaggagggcgagctgtctttctatgacgcggagcgccactgccacctgtacaccttccacgcccgctttggggaggttcgcccctacttctacctggggggtgcacggggcgccgggcctccagagcctttgcgcatctgccccttgcacatcagtgtcaaggaagaactggatggctgagctggcccggggctgccccggtcttgtgccacagcactgttttctttctgccctcttcctaatgcccacactgcttgggcactattgcgcccctgcctccttgccaggctcttcctcctgtcctgcctggtccttttccatgactccaggctgtgcctctctccatgtttggtcccttctgtgcccatggtcaggagctattcgggtggcacctcgctggccaggctctcccgagtcgtggcacctccacaatgtgaattttctgaatccctattccaggatttctgggaataatgtttacttctagaatgggcctgttgtaaaccatctcatcgaggtgtggtaaagccattggatgaggaggggactgccatggaaaggagagtttgttacttacggttctgagaggaggggccacataggaaagccccacggtgggtcagaaggcggaaggagggaggggaacgtgtgggcaagagacttcctctggtttcctcaggaggaaatgggcaaggcagagtaagcaggggagacaggtttaagggtagctggcttgagtaatttcagtggctctcaggataggggctgccctttttgtctgatacctggccccgggatagtcaggacaggtgaatgttggcctggggtgtgacagccctgggagagccatgtgaaggaggcagctggcgccatcgctccggattagttggtttccataggaaaggcatgctttcagccagatgcttgccatctctagggattgggggattggctagcctgggaggatcagtctgtccaggtcagcgaggccccagataccagagcatcaagagtacaggaaatacagttaatgcagggcctctgtgtggctggatcctccgtctccatcagatcagctctgattgatctattcttgcacgatttcctctgaacacagggttccagagtacttaaacacaacattttttaaatcgtgatttcggcctatttccttgccaggcctgtttccccaccaggaaatgagataggaggactggatgaggatgtcctgttatagttgctgtggaggaagttcctctggttaattctcatcagcgtctgcagaaaagaaggaaagagggcacccttttcagttgggaagaaaggagaggggtggcgccatggacgtggccctaaacgctgtgggagagggaagaggaggctgggcctcgctgccctcttgtctctgctgacttcagcctggtcatgcttgctctgccacttgcgatttcatccctaatttcttcctccaccatgcctgcagacttttccctgggcttgttttttctcgcacatctctgaagagtttttaatcttcagctcatcatgtcccaggaagtggcatcataaaaggaaatatttttttttcctaggagcagtgttaaaatctgggtcacattcctgaccaaggacagcatcctgccttttggccatccccttcagttcacaaaagctgacattttaaacaaatcatgactcacacgtattaattggttataaatatgttgtgtacactggttagataaaacttaaggccacaaggagggcccaggtaggcgatgtcagtgtgtgaaggggctggattgggcgtggtgaggatgttggcaaaccagtgcatgcacctggttggaagatgctcagcctcacaaaagctccaagccctttgggagccaaagtgtctgagagtgtgaccctctcctgtaaagtatttatcccacccattaatataatttctgtataataaacttgacctgaaattatttcattctttatattaaacttttaaaaatgttttttattttcaccttagatatgggaagagttttttttttttttttttttttaacaggataacttgagcaggctaggcctcttaaaaaaaaatttgagctaaaactcatttttcttttggcattttcttttcaatgttcttataagcaaagttcatccatgttgtagcatgtgttcaactttattttttcatcgggtaatattccattgtatggaatggtagtactacattttatttatcatgcatcgattggtggacatttggatcgtttctacttcttgactattatacataatgctgctaggaacttttgtgtatgagtttttgtgtggacatatgttttcatttctctttggtatatgcctgggagcagatttgctggatcatatgaaactctatttaacccttgagggactcccaaactgttttccaatgtggtgacacaattttatatcccatcaacagggcatgagggttctgatgactccacatccctcagtgctttttattatctatcttttaacttagccatcctagtaggggtaatgtggcatctcattgtgattttggtttgcatttccctgatggcgaatgatattgagcatcttttcatgagcttattggccatttgcatatcttcttaggacagctatctttagatcacttgctcattttttaattgggttatttgtctttttattattgagttgtaagagtccttttatagcctggcacaagtccctttaactggtatatgattataaaatttttctccgtgagctgtttcatttccttgatgatgtcctttgaaatactaaagcttttaattttg

SEQ ID NO: 6 - protein, 206 aa, Homo sapiens
MERSPDVSPGPSRSFKEELLCAVCYDPFRDAVTLRCGHNFCRGCVSRCWEVQVSPTCPVCKDRASPADLRTNHTLNNLVEKLLREEAEGARWTSYRFSRVCRLHRGQLSLFCLEDKELLCCSCQADPRHQGHRVQPVKDTAHDFRVRSLIAEERRNFLPTHQWIVTKTRLQTSSPNLQSRRQGQVQEHGACTAGEGQGLLGHAALL

SEQ ID NO: 7 - protein, 192 aa, Mus musculus
MEPGPSVSPGPSRSFKEELLCAVCYDPFRDAVTLRCGHNFCRRCVSGCWEVQTTPSCPVCKERAVPGELRTNHTLNNLVETLLREEAEGARWTGRRSPRPCRAHRAPLTLFCLEDKELLCCACQADARHQEHRVQPIKDTAQDFRAKCKNMEHVLREKAKAFWALRRTYEAIAKHNEVQTTWLEGRIRDEFD

SEQ ID NO: 8 - protein, 309 aa, Mus musculus
KLRDFLRVEEQATLDAMKEESRKKHLQAEEKMKQLAEQTEALAREIERLQMEMKEDDMTFLMKHKSRKRRLFCTVEPAPLQPGLLMDACKYLESLQYRVWKKMLGSVESVPFSLDPNTAAGWLKVADDLTSVINHGYRVQVENPERFSSAPCLLGSQVFSKGSHSWEVDVGGLPTWRVGVVRVQAHAQAQAQADVGGEGHSHSCYHDTRSGFWYLCRTQGVDGDHCMTSDTATAPLVQAMPRRLRVELECEEGELSFYDSERHCHLYTFHAHFGEVRPYFYLGASRGDGPPEPLRICHLRVSIKEELDI

SEQ ID NO: 9 - DNA, 22 bp, Artificial
  Primer sequence for Amplification of HLS-5
ttcggctcttgtgtttcatgag

SEQ ID NO: 10 - DNA, 18 bp, Artificial
  Primer for amplifciation of HLS-5
tcaggtggaggctgcatg

(19) United States
(12) Patent Application Publication
(10) Pub. No.: US 2010/0215637 A1
(43) Pub. Date: Aug. 26, 2010

(54) Agent for the Treatment of Hormone-Dependent Disorders and Uses Thereof

(75) Inventors: S. Peter Klinken, Western Australia (AU); Jean-Phillippe Lalonde, Western Australia (AU)

Correspondence Address: K&L Gates LLP, STATE STREET FINANCIAL CENTER, One Lincoln Street, BOSTON, MA, 02111-2950, US

(73) Assignee: Biopharmica Ltd, North Perth, Western Australia (AU)

(21) Appl. No.: 12/294,812

(22) PCT Filed: Mar. 28, 2007
(86) PCT No.: PCT/AU2007/000399

§ 371 Date: Nov. 21, 2008

Related U.S. Application Data
(60) Provisional application No. 60/787,283, filed on Mar. 28, 2006.

(30) Foreign Application Priority Data
Nov. 21, 2006 (AU) 2006906505

(51) Int. Cl. A61K 39/395; A61K 38/16; A61K 31/7088; G01N 33/567; A61P 25/28; A61P 35/00

(52) U.S. Cl. 424/130.1; 514/12; 514/44 R; 514/44 A; 435/7.21

(57) ABSTRACT

The present invention relates to novel agents that are useful for treating hormone-dependent disorders. In particular, the present invention relates to A method of modulating a nuclear receptor in vivo comprising the step of administering to a subject in need thereof A nuclear receptor modulating agent comprising a compound or composition capable of altering the endogenous levels of HLS-5 or its activity.

### PRIORITY CLAIM

This application claims the benefit under 35 USC 119(e) of U.S. provisional application No. 60/787,283 filed on Mar. 28, 2006, the contents of which are incorporated herein by reference.

### FIELD

The present invention relates to novel agents that are useful for treating hormone-dependent disorders. In particular, the present invention relates to a nuclear receptor modulating agent comprising either a pharmaceutically-effective amount of a HLS-5 polypeptide or functional fragment thereof or pharmaceutical composition thereof; or a compound or composition capable of modulating the endogenous levels of HLS-5 or its activity, wherein said agent is effective in the treatment of a hormone-dependent disorder.

### BACKGROUND

The hormone-dependent disorders are a broad class of conditions with one common feature: they are all linked to nuclear receptor status. There are a number of hormone dependent disorders including, but not limited to, androgen-dependent and independent prostate cancer, breast cancer, osteoporosis, Alzheimer's disease, cerebrovascular disease, pre-eclampsia and endometriosis, hyper and hypothyroidism related diseases with or without malignant or autoimmune component, Cushing's syndrome or adrenal hyper or hypofunction.

One particular group of hormone-dependent disorders has been studied much more than others and that is hormone-dependent cancers. Breast and prostate cancer are the most classic examples of hormone-dependent cancers. In the case of breast cancer the hormone oestrogen (E2) regulates the growth of the majority of breast cancers, while prostate cancer is generally regulated by androgens or dihydroxytestosterone (DHT). Other examples of hormone-dependent cancer include endometrial cancer, ovarian cancer, thyroid cancer and prostate cancer (See, for example, Sherman et al. 2000, Rev Endocr Metab Disord. 1, 165-71; Makar et al., 2000, Endocr Relat Cancer. 7, 85-93; Schroder et al., 1999, BJU Int., 83, 161-70; Chen et al., 1999, Oncology. 13, 1665-70; Feldman & Feldman, 2001, Nature Reviews Cancer, 1, 34-45.

Breast and prostate cancer are amongst the leading causes of cancer death in women and men, respectively, in most industrialized countries. Being hormone-dependent tumors, anti-hormone therapies usually are effective in prevention and treatment. However, the emergence of resistance is common, especially for locally advanced tumors and metastatic tumors, in which case resistance is predictable. The phenotypes of these resistant tumors include receptor-positive, ligand-dependent; receptor-positive, ligand-independent; and receptor-negative, ligand-independent. The underlying mechanisms of these phenotypes are complicated, involving not only sex hormones and sex hormone receptors, but also several growth factors and growth factor receptors, with different signalling pathways existing alone or together, and with each pathway possibly linking to one another.

With respect to breast cancer, the majority of patients present with localised disease, also known as primary breast cancer. The usual treatment is surgical excision of the tumour, followed by adjuvant therapy. Adjuvant therapies for oestrogen receptor α (ERα)-positive disease are designed to reduce oestrogen levels or block its activity by binding to the receptor, as exemplified by Tamoxifen™. Tamoxifen™ is now the first line adjuvant treatment for ERα-positive disease in pre- and post-menopausal women and is beneficial in the treatment of metastatic disease, as well as localized disease. However, approximately 30%, of patients with ERα-positive disease do not respond to Tamoxifen™. Moreover, a substantial proportion of patients presenting with localized disease and all patients presenting with metastatic disease that initially respond to Tamoxifen™ treatment become resistant. In the case of patients who initially respond, but become resistant to Tamoxifen™, ERα expression is lost in only about 10% of cases. Moreover, one-third of resistant patients show a clinical response to treatment with a different anti-estrogen such as Faslodex™ (ICI 182, 780) or the use of aromatase inhibitors, drugs that inhibit estrogen synthesis. Nevertheless and despite issues of de novo or acquired resistance to Tamoxifen™, it has become the adjuvant agent of first choice (Ali & Coombes, 2002, Nature Reviews Cancer, 2: 101-112).

Several factors, both inducers and inhibitors, play essential roles in the regulation of oestrogen responsive genes. The nuclear hormone receptor transcription factors (NRs) respond directly to small-molecule ligands and control diverse functions in vivo, including development and dynamic homeostasis (Aranda & Pascual, 2001, Physiol Rev, 81, 1269-1304; Tsai & O'Malley, 1994, Annu Rev Biochem, 63, 451-486). Both metabolic diseases and cancer have been directly correlated with the mis-regulation of signalling by the NRs, and they are ideal target for multiple drugs and drug development programs. Ligand-dependent NR signalling requires direct interaction between NRs and the steroid receptor coactivators (SRCs), effected by a conserved SRC motif (NR box, L₁XXL₂L₃) (Tsai & O'Malley, 1994, supra).

Estrogen receptors (ERα and ERβ) mediate the effects of 17beta-estradiol (E2) and account for E2 role on growth, development, and homeostasis maintenance in different tissues and organs. ERα and ERβ function as ligand-dependent transcription factors which directly bind to specific estrogen responsive element (ERE) present into DNA and, in turn, regulate the transcription of E2-sensitive genes. In addition, ERα and ERβ, without direct binding to DNA, regulate transcription indirectly by binding to other transcription factors activating or inactivating the transcription of E2-dependent-ERE-devoid genes. Along with these two E2 mechanisms, it has been recently uncovered that a third signalling pathway, involving cytoplasmic proteins and rapid membrane-initiated responses, serves largely for mitogenic E2-induced effects. The commitment of ERβ in these rapid E2-induced effects is openly debated.

With respect to prostate cancer it often develops from clones that are already present as early as thirty-five years of age, when circulating concentrations of active androgens or Dihydroxytestosterone (DHT) are high. The progression of the disease is slow and the cancer is diagnosed at a more advanced age. Prostate cancer evolves from an androgen dependant stage to stage where it escapes from all anti-androgenic treatments (Petrylak, 2005, Urology, 65:6, p8-12). The patient usually dies within two years following the diagnosis of advanced cancer. Therefore, it is of great interest to develop new therapies for androgen independent prostate cancer. The androgen independent evolution of prostate cancer is a complex phenomenon at the cellular and molecular levels. It includes an increased sensitivity to growth factors, the control of proliferation pathways, apoptotic and survival pathways as well as the control of angiogenesis.

Transformation and progression towards malignancy in prostate cancer is partly dependant on the inability of the prostatic epithelial cells to undergo apoptosis rather than on the regulation of proliferation. Molecular targeting of inadequacies in this process of suppression of apoptosis could prove to be of great therapeutic importance for prostate cancer patients.

Accordingly, it would be useful to find other agents capable of preventing and/or treating hormone-dependent disorders per se or locating agents capable of assisting the action of “known” agents.

### SUMMARY

The present inventors have found that HLS-5 is capable of affecting and/or modulating the action and activity of nuclear receptors, especially those nuclear receptors associated with hormone-dependent disorders.

Accordingly, in a first aspect the present invention provides a nuclear receptor modulating agent comprising a compound or composition capable of altering the endogenous levels of HLS-5 or its activity.

In a second aspect the present invention provides a nuclear receptor modulating agent comprising either:

(i) a pharmaceutically-effective amount of a HLS-5 polypeptide or an isoform or functional fragment thereof or pharmaceutical composition thereof; or

(ii) a compound or composition capable of altering the endogenous levels of HLS-5 or its activity; or combinations thereof,

wherein the agent is effective in the treatment of a hormone-dependent disorder.

In some embodiments, the agent further comprises an HLS-5 polypeptide. In some embodiments, the HLS-5 polypeptide will comprise the sequence set out in SEQ ID NO:2, SEQ ID NO:4 or SEQ ID NO:6 or a polypeptide substantially homologous thereto, or a functional fragment thereof.

It is to be clearly understood that the HLS-5 polypeptide of the present invention also includes polypeptide analogues, including but not limited to the following:

1. HLS-5 polypeptide in which one or more amino acids is replaced by its corresponding D-amino acid. The skilled person will be aware that retro-inverso amino acid sequences can be synthesised by standard methods. See for example Chorev and Goodman, 1993, Acc Chem Res, 26: 266-273;
2. Peptidomimetic compounds of HLS-5, in which the peptide bond is replaced by a structure more resistant to metabolic degradation. See, for example, Olson et al, 1993, J. Med. Chem., 36, p3039-3049.
3. HLS-5 polypeptide in which individual amino acids are replaced by analogous structures, for example gem-diaminoalkyl groups or alkylmalonyl groups, with or without modified termini or alkyl, acyl or amine substitutions to modify their charge.

The use of such alternative structures can provide significantly longer half-life in the body, since they are more resistant to breakdown under physiological conditions.

Methods for combinatorial synthesis of polypeptide analogues and for screening of polypeptides and polypeptide analogues are well known in the art (see, for example, Gallop et al., 1994, J. Med. Chem., 37, p1233-1251). It is particularly contemplated that the HLS-5 polypeptides of the invention are useful as templates for design and synthesis of compounds of improved activity, stability and bioavailability.

Preferably where amino acid substitution is used, the substitution is conservative, i.e., an amino acid is replaced by one of similar size and with similar charge properties.

In some embodiments, the HLS-5 polypeptide will be expressed in vivo from a vector comprising a polynucleotide encoding HLS-5. In some embodiments, the HLS-5 polynucleotide will be selected from the group consisting of:

(a) polynucleotides comprising the nucleotide sequence set out in SEQ ID NO:1, SEQ ID NO:3 or SEQ ID NO:5, or a functional fragment thereof;

(b) polynucleotides comprising a nucleotide sequence capable of hybridizing selectively to the nucleotide sequence set out in SEQ ID NO:1, SEQ ID NO:3 or SEQ ID NO:5, or a functional fragment thereof;

(c) polynucleotides comprising a polynucleotide sequence which is degenerate as a result of the genetic code to the polynucleotides defined in (a) or (b);

(d) polynucleotides complementary to the polynucleotides of (a), (b) or (c).

The present invention also provides a vector comprising a HLS-5 polynucleotide of the invention, for example an expression vector comprising a HLS-5 polynucleotide of the invention, operably linked to regulatory sequences capable of directing expression of said polynucleotide in a host cell.

Accordingly, in a third aspect, the present invention provides a nuclear receptor modulating agent comprising a mammalian expression vector comprising a HLS-5 polynucleotide of the invention, operably linked to a regulatory sequence capable of directing expression of said polynucleotide in a host cell.

In a fourth aspect the present invention provides a method of modulating a nuclear receptor in vivo comprising the step of administering to a subject in need thereof:

(i) an effective amount of a HLS-5 polypeptide, isoform thereof, functional fragment thereof or composition thereof; or

(ii) a substance capable of altering the endogenous level and/or activity of HLS-5 or an isoform or functional modification or functional fragment thereof; or

(iii) an antibody or fragment thereof which specifically binds HLS-5 or an isoform or functional modification or functional fragment thereof; or

(iv) a polynucleotide molecule which is antisense to a polynucleotide molecule which encodes HLS-5 or an isoform or functional modification or functional fragment thereof; or

(v) combinations thereof.

In a fifth aspect, the present invention provides the use of one or more of:

(i) a pharmaceutically-effective amount of a HLS-5 polypeptide, isoform thereof, functional fragment thereof or composition thereof; or

(ii) a substance capable of altering the endogenous level and/or activity of HLS-5 or an isoform or functional modification or functional fragment thereof; or

(iii) an antibody or fragment thereof which specifically binds HLS-5 or an isoform or functional modification or functional fragment thereof; or

(iv) a polynucleotide molecule which is antisense to a polynucleotide molecule which encodes HLS-5 or an isoform or functional modification or functional fragment thereof; or

(v) combinations thereof

for modulating a nuclear receptor in vivo.

In some embodiments, the nuclear receptor modulating agent will down-regulate the activity of the nuclear receptor by either direct or indirect means. In yet other embodiments, the nuclear receptor modulating agent will up-regulate the activity of the nuclear receptor.

In a sixth aspect the present invention provides a method of modulating a nuclear receptor in vitro comprising the step of administering to cells:

(i) an effective amount of a HLS-5 polypeptide, isoform thereof, functional fragment thereof or composition thereof; or

(ii) a substance capable of altering the endogenous level and/or activity of HLS-5 or an isoform or functional modification or functional fragment thereof; or

(iii) an antibody or fragment thereof which specifically binds HLS-5 or an isoform or functional modification or functional fragment thereof; or

(iv) a polynucleotide molecule which is antisense to a polynucleotide molecule which encodes HLS-5 or an isoform or functional modification or functional fragment thereof; or

(v) combinations thereof.

In a seventh aspect the present invention provides a method for treating or preventing a hormone-dependent disorder comprising the step of administering to a subject in need thereof:

(i) a pharmaceutically-effective amount of a HLS-5 polypeptide, isoform thereof, functional fragment thereof or pharmaceutical composition thereof; or

(ii) a substance capable of altering the endogenous level and/or activity of HLS-5 or an isoform or functional modification or functional fragment thereof; or

(iii) an antibody or fragment thereof which specifically binds HLS-5 or an isoform or functional modification or functional fragment thereof; or

(iv) a polynucleotide molecule which is antisense to a polynucleotide molecule which encodes HLS-5 or an isoform or functional modification or functional fragment thereof; or

(v) combinations thereof.

In some embodiments the disorder will be directly affected by or controlled by the nuclear receptor modulating agent. In other embodiments, the disorder will not be directly affected by or controlled by the nuclear receptor modulating agent; however, the administration of the nuclear receptor modulating agent improves, alleviates or treats the disorder by modulating nuclear receptors of polypeptides that are associated with or affected by the disorder.

The nuclear receptor modulating agents of the invention may be administered by any suitable route, and the person skilled in the art will readily be able to determine the most suitable route and dose for the condition to be treated. Dosage will be at the discretion of the attendant physician or veterinarian, and will depend on the nature and state of the condition to be treated, the age and general state of health of the subject to be treated, the route of administration, and any previous treatment which may have been administered.

The nuclear receptor modulating agent may be administered in the form of a composition further comprising a pharmaceutically acceptable carrier. This will usually comprise at least one excipient, for example selected from the group consisting of sterile water, sodium phosphate, mannitol, sorbitol, sodium chloride, and any combination thereof.

Methods and pharmaceutical carriers for preparation of pharmaceutical compositions are well known in the art, as set out in textbooks such as Remington's Pharmaceutical Sciences, 20th Edition, Williams & Wilkins, Pennsylvania, USA.

The carrier or diluent, and other excipients, will depend on the route of administration, and again the person skilled in the art will readily be able to determine the most suitable formulation for each particular case.

The subject may be a human, or may be a domestic, companion or zoo animal. While it is particularly contemplated that the nuclear receptor modulating agents of the invention are suitable for use in medical treatment of humans, they are also applicable to veterinary treatment, including treatment of companion animals such as dogs and cats, and domestic animals such as horses, cattle and sheep, or zoo animals such as non-human primates, felids, canids, bovids, and ungulates.

In an eighth aspect the present invention provides, an assay for identifying a modifier of HLS-5 nuclear receptor modulating activity, comprising:

(i) providing a nuclear receptor test system including a responsive element capable of binding HLS-5 or fragment thereof, under conditions which permit the binding of HLS-5 or fragment thereof to the responsive element;

(ii) contacting the nuclear receptor test system with HLS-5;

(iii) measuring the level of interaction in the presence of the HLS-5;

(iv) contacting the nuclear receptor system with a candidate modifier of HLS-5 nuclear receptor modulating activity;

(v) measuring the level of interaction in the presence of the candidate modifier; and

(iv) comparing the measured level of interaction in the presence of the candidate modifier with the measured level of interaction in the presence of HLS-5 and/or absence of the candidate modifier, wherein a statistically significant alteration in the interaction in the presence of the candidate modifier is indicative of a modifier of HLS-5 nuclear receptor modulating activity.

In some embodiments, the nuclear receptor test system comprises a cell having a reporter gene construct comprising a HLS-5 responsive element and a reporter gene operably linked thereto. Preferably, the reporter gene is a fluorescent protein or an enzymatic reporter. More preferably, the reporter gene is selected from the group consisting of green fluorescent protein, luciferase, beta-galactosidase, beta-glucuronidase, beta-lactamase, horseradish peroxidase, alkaline phosphatase and chloramphenicol acetyl transferase (CAT).

In a ninth aspect the present invention provides a method of determining the suitability of HLS-5 treatment of a hormone dependent disorder comprising:

(i) providing a biological sample from a test subject;

(ii) measuring the level of nuclear receptor activity in the biological sample; and

(iii) contacting the biological sample with HLS-5 or functional fragment thereof and measuring the level of nuclear receptor activity and comparing the measured level of nuclear receptor activity in the presence of the HLS-5 with the nuclear receptor activity in the absence of HLS-5, wherein a statistically significant difference in nuclear receptor activity in the presence of the HLS-5 is indicative of the suitability of HLS-5 treatment of the condition.

In a tenth aspect, the present invention provides a microarray comprising one or more of:

(i) HLS-5 or an isoform or functional modification or functional fragment thereof;

(ii) a polynucleotide molecule encoding HLS-5 or an isoform or functional modification or functional fragment thereof;

(iii) a substance capable of altering the endogenous level and/or activity of HLS-5 or an isoform or functional modification or functional fragment thereof;

(iv) an antibody or fragment thereof which specifically binds HLS-5 or an isoform or functional modification or functional fragment thereof; and/or

(v) a polynucleotide molecule which is antisense to a polynucleotide molecule which encodes HLS-5 or an isoform or functional modification or functional fragment thereof.

In an eleventh aspect, the present invention provides a kit comprising one or more of:

(i) HLS-5 or an isoform or functional modification or functional fragment thereof;

(ii) a polynucleotide molecule encoding HLS-5 or an isoform or functional modification or functional fragment thereof;

(iii) a substance capable of altering the endogenous level and/or activity of HLS-5 or an isoform or functional modification or functional fragment thereof;

(iv) an antibody or fragment thereof which specifically binds HLS-5 or an isoform or functional modification or functional fragment thereof; and/or

(v) a polynucleotide molecule which is antisense to a polynucleotide molecule which encodes HLS-5 or an isoform or functional modification or functional fragment thereof.

### BRIEF DESCRIPTION OF THE FIGURES

FIG. 1 shows the level of HLS5 in cancer cell lines of different origins.

FIG. 2 shows that HLS5 mRNA is widely suppressed in primary breast and ovarian cancers. Individual tumours with their normal surrounding tissues have been labelled from A to E.

FIG. 3 shows that HLS5 mRNA is partially induced in primary colon and lung cancers. Individual tumours with their normal surrounding tissues have been labelled from A to E.

FIG. 4 shows analysis of possible epigenetic modification of HLS5. Panel (A) shows the region within the HLS5 promoter and first exon which is likely to be a CpG island using the The CpG Island Searcher (http://www.bioinfo.de/isb/2003/03/0021/main.html). Panel (B) shows the 5-azacytidine-induced re-expression of HLS5 in a MDA-MB-435 cell line.

FIG. 5 shows that HLS5 affects the activity of both androgen and oestrogen receptors.

FIG. 6 shows that HLS5 inhibited the transcriptional activity by the androgen and oestrogen receptor in a dose-dependent manner.

FIG. 7 shows mapping the domains responsible for HLS5 transcriptional repression activity.

FIG. 8 shows the identification of minimal receptor interaction domains between hSKIP and in HLS5.

FIG. 9 shows the identification of minimal receptor interaction domains between TDG and in HLS5.

### DETAILED DESCRIPTION OF THE INVENTION

Before describing the present invention in detail, it is to be understood that this invention is not limited to particularly exemplified HLS-5 sequences, expression techniques or methods and may, of course, vary. It is also to be understood that the terminology used herein is for the purpose of describing particular embodiments of the invention only, and is not intended to be limiting which will be limited only by the appended claims.

All publications, patents and patent applications cited herein, whether supra or infra, are hereby incorporated by reference in their entirety. However, publications mentioned herein are cited for the purpose of describing and disclosing the protocols and reagents which are reported in the publications and which might be used in connection with the invention. Nothing herein is to be construed as an admission that the invention is not entitled to antedate such disclosure by virtue of prior invention.

The practice of the present invention will employ, unless otherwise indicated, conventional techniques of cell biology, cell culture, molecular biology, recombinant DNA, pharmacology and immunology, which are within the skill of the art. Such techniques are described in the literature. See, for example, Bailey & 011 is, 1986, “Biochemical Engineering Fundamentals”, 2nd Ed., McGraw-Hill, Toronto; Coligan et al., 1999, “Current protocols in Protein Science” Volume I and II (John Wiley & Sons Inc.); “DNA Cloning: A Practical Approach”, Volumes I and II (Glover ed., 1985); Handbook of Experimental Immunology, Volumes I-IV (Weir & Blackwell, eds., 1986); Immunochemical Methods in Cell and Molecular Biology (Mayer and Walker, eds., Academic Press, London, 1987), Methods in Enzymology, Vols. 154 and 155 (Wu et al., eds. 1987); “Molecular Cloning: A Laboratory Manual”, 2^{nd} Ed., (ed. by Sambrook, Fritsch and Maniatis) (Cold Spring Harbor Laboratory Press: 1989); “Nucleic Acid Hybridization”, (Hames & Higgins eds. 1984); “Oligonucleotide Synthesis” (Gait ed., 1984); Remington's Pharmaceutical Sciences, 17^{th} Edition, Mack Publishing Company, Easton, Pa., USA.; “The Merck Index”, 12^{th} Edition (1996), Therapeutic Category and Biological Activity Index; and “Transcription & Translation”, (Hames & Higgins eds. 1984).

It must be noted that as used herein and in the appended claims, the singular forms “a,” “an,” and “the” include plural reference unless the context clearly dictates otherwise. Thus, for example, a reference to “a nucleic acid molecule” includes a plurality of such molecules, and a reference to “an agent” is a reference to one or more agents, and so forth. Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

The present invention encompasses the following aspects: preparation of a nuclear receptor modulating agent of the present invention; preparation of the polynucleotide encoding said agent or a recombinant vector carrying and expressing said polynucleotide; transformants carrying said vector; methods of producing said transformants; methods of detecting the nuclear receptor modulating agent; methods of detecting the mRNA or polynucleotide encoding said agent; and methods of treating conditions caused by or exacerbated by unregulated nuclear receptor activity are explained below.

In the description that follows, if there is no instruction, it will be appreciated that techniques such as gene recombinant techniques, production of recombinant polypeptides in animal cells, insect cells, yeast and Escherichia coli, molecular-biological methods, methods of separation and purification of expressed HLS-5 polypeptides, assays and immunological methods, are well-known in this field and any such technique may be adopted.

In its broadest aspect the present invention provides a nuclear receptor modulating agent comprising an effective amount of a HLS-5, isoform thereof or functional fragment thereof.

The term “nuclear receptor modulating agent” as used herein refers to a compound or composition of matter comprising HLS-5, isoform, functional fragment or derivative thereof that is capable of modulating directly or indirectly the activity of a nuclear receptor. The terms “modulate,” “modulating,” or “modifier” are used herein interchangeably to refer to actions of HLS-5, isoform thereof, functional fragment or derivatives thereof to alter or affect (e.g., either up-regulate i.e. increase the function and/or expression, down-regulate i.e. decrease the function and/or expression, inhibit, antagonize, agonize, induce, or suppress or otherwise control) one or more nuclear receptor(s).

Assays described herein (see Examples) and otherwise known in the art may routinely be applied to measure the ability of the nuclear receptor modulating agent of the present invention and fragments, variants and derivatives thereof to elicit an effect eg modulate or modify the nuclear receptors on cells. For example, in some embodiments the ability of the nuclear receptor modulating agent to bind to the nuclear receptor or compete with full-length HLS-5 polypeptide can be assessed by various immunoassays known in the art. Assays that can be used include, but are not limited to, competitive and non-competitive assay systems using techniques such as radioimmunoassays, ELISA (enzyme linked immunosorbent assay), “sandwich” immunoassays, immunoradiometric assays, gel diffusion precipitation reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. All of these assays can utilise anti-nuclear receptor antibodies.

Methods for assessing the level of nuclear receptor expression in a cell and/or tissues, including Northern blot analysis, qPCR, and the like, are disclosed herein or are well known to those skilled in the art. Such assays can be used to determine the effect of the nuclear receptor modulating agent on the nuclear receptor.

As the nuclear receptor modulating agent of the present invention is capable of modifying the nuclear receptor activity of hormonally active receptors (particularly estrogen or androgen) it is possible to monitor the oestrogen receptor (ERalpha and ERbeta) protein levels in tissues and cell lines by western blot and immunohistochemistry. Northern blot analysis or qPCR can show control at the mRNA levels in response to nuclear receptor modulating agent, as well as when these tissues are treated with 17beta-estradiol (E2).

Another assay that can be used in the present invention to assess the level of modulation is the reporter gene assay. As used herein, the term “reporter gene” means a gene, the expression of which can be detected using a known method. There are a number of well characterised reporter genes including the genes for fluorescent proteins, such as green fluorescent protein, a luciferase, a beta-galactosidase, a beta-glucuronidase, a beta-lactamase, a peroxidase such as horseradish peroxidase, an alkaline phosphatase, CAT, etc.). Using reporter plasmids containing androgen response elements derived from the human secretory component or the rat probasin genes as well as the prostate specific antigen consensus response element and in the case of the oestrogen receptor, the oestragen response element. It is possible to compare the activity of these responsive elements to the nuclear receptor modulating agent of the present invention. Cells transiently transfected with human cDNA constructs or soluble compounds can be assayed. The potency of testosterone or respective ligands can be tested in the same manner. This provides a highly sensitive in vitro test system to analyze endocrine activities of transcriptional regulators.

In some embodiments, a useful reporter gene assay is one utilising the glucocorticoid response elements (“GRE”) or thyroid receptor enhancer-like DNA sequence (“TRE”). GRE's are enhancer-like DNA sequences that confer glucocorticoid responsiveness via interaction with the GR. See Payvar et al., 1983, Cell, 35:381 and Schiedereit et al., 1983, Nature, 304:749. TREs that function preferentially in prostate cells include, but are not limited to, TREs derived from the prostate-specific antigen gene (PSA-TRE) (U.S. Pat. No. 5,648,478), the glandular kallikrein-1 gene (from the human gene, hKLK2-TRE), and the probasin gene (PB-TRE) (International Patent Application No. PCT/US98/04132). All three of these genes are preferentially expressed in prostate cells and the expression is androgen-inducible. Generally, expression of genes responsive to androgen induction requires the presence of an androgen receptor (AR).

Another useful assay for monitoring the affect of the nuclear receptor modulating agents of the present invention, is the chromatin immunoprecipitation (ChIPs) assay. Preferably, the procedure utilised is one of the methods described by Das et al., 2004, Biotechniques, 37(6): p. 961-9 or Metivier et al., 2006, EMBO Rep, 7(2): p. 161-7. Basically, cells can be grown to 50-60% confluency and crosslinked with a 1% formaldehyde solution in 1×PBS for 15 min at room temperature. Crosslinking is stopped by addition of glycine to 125 mM final concentration. Monolayers are then washed twice with cold PBS and harvested in radioimmunoprecipitation assay (RIPA) buffer [150 mM NaCl, 1% v/v Nonidet P-40, 0.5% w/v deoxycholate, 0.1% w/v SDS, 50 mM Tris pH 8, 5 mM EDTA, protease inhibitor cocktail, phosphatase inhibitors (20 mM NaF/0.2 mM sodium orthovanadate), deacetylase inhibitors (5 μM trichostatin A/5 mM sodium butyrate) and 0.5 mM PMSF] to generate a suspension of 10 million cells/ml. Suspensions are then sonicated to generate DNA fragments below 500 by and clarified by centrifugation for 10 min at 12000 g. Protein solutions can then be quantified for protein and DNA content and adjusted to 1 mg/ml of protein.

For immunoprecipitation, 1 mg of protein extract is pre-cleared for 2 hrs with 40 μl of a 50% slurry of 50:50 proteinA:proteinG sepharose before addition of indicated antibodies. Antibody (Ab) sources are then added and incubated for 12 hrs at 4° C. in the presence of 40 μl of a 50% slurry of 50:50 proteinA:proteinG sepharose pre-blocked with 1 mg/ml bovine serum albumin (BSA) and 0.3 mg/ml of sonicated salmon sperm DNA. Sepharose beads are recovered by centrifugation for 1 min at 6000 g and washed twice with RIPA buffer, four times with ChIP Wash Buffer [100 mM Tris HCl pH 8.5, 500 mM LiCl, 1% v/v Nonidet P-40, 1% w/v deoxycholic acid], twice with RIPA buffer and twice with 1×TE. Immunocomplexes are eluted for 10 min at 65° C. in the presence of 1% SDS and crosslinking is reversed by adjusting to 200 mM NaCl and incubating 5 hrs at 65° C. Samples are treated with Proteinase K followed by phenol-chloroform extraction. DNA is precipitated and used as template in semi-quantitative PCR reactions.

Other assays that could be used to assess the degree of modulation include electrophoretic mobility shift assay or EMSA (See, for example, Crothers, 1998, Nature, 325: 464-5; Garner & Revzin, 1986, Trends in Biochemical Sciences, 11: 395-6; Hendrickson, 1985, BioTechniques, 3:198-207; Buratowski & Chodosh, in Current Protocols in Molecular Biology pp. 12.2.1-12.2.7 (Ausubel ed., 1996); see also U.S. Pat. No. 5,789,538, co-owned WO 00/42219 herein incorpoated by reference in its entirety.

The gene for HLS-5 or Hemopoietic Lineage Switch 5 is found on chromosomal loci 8p21.1, a region often deleted in cancers. Deletions to this locus have been implicated in breast, ovarian, prostate, colorectal and liver cancers (Armes et al., 2004, Oncogene, 23, 5697-5702; Brown at al., 1999, Gynecol Oncol, 74, 98-102; Bruix & Llovet, 2002, Hepatology, 35, 519-524; Chughtai et al., 1999, Oncogene, 18, 657-665; Courtay-Cahen et al., 2000, Genomics, 66, 15-25; Nihei et al., 2002, Cancer Res, 62, 367-370; Oba at al., 2001, Cancer Genet Cytogenet, 124, 20-26; Seitz et al., 1997, Oncogene, 14, 741-743; Wright et al., 1998, Oncogene, 17, 1185-1188).

HLS-5 is a member of the RING finger B-box Coiled-coil (RBCC) protein family (Lalonde at al., 2004, J Biol Chem, 279, 8181-8189). This group of molecules is also referred to as the tripartite motif family (TRIM) of proteins, because of the characteristic domain architecture that is conserved amongst higher eukaryotes (Reymond et al., 2001, Embo J, 20, 2140-2151). Sequence analysis of the mouse and human genomes has identified a diverse array of RBCC proteins, many with unknown functions (Reymond et al., 2001, supra). Several RBCC family members, including PML, TIFla and Rfp, are mutated in human cancer, implicating RBCC proteins as crucial regulators of cell growth and differentiation (de The et al 1991, Cell, 66:675-684). Recent studies have demonstrated that some RBCC members regulate the activity, or steady-state levels, of partner proteins by influencing subcellular localization or post-translational modifications (Diamonti et al., 2002, Proc Natl Acad Sci USA, 99, 2866-2871; Pearson et al., 2000, Nature, 406, 207-210; Urano et al., 2002, Nature, 417, 871-875).

HLS-5 was originally identified as a gene markedly up-regulated during an erythroid to myeloid lineage switch of the J2E erythroid cell line (Klinken at al., 1988, Proc. Natl. Acad. Sci., USA, 85, 8506-8510; Lalonde et al., 2004, supra). The myeloid variants displayed a monoblastoid morphology, did not respond to erythropoietin (EPO) and had reduced expression of erythroid-specific transcription factors, including GATA-1 and EKLF (Keil et al., 1995, Cell Growth Differ., 6, 439-448; Williams et al., 1999, Embo J., 18, 5559-5566). Significantly, HLS-5 was isolated independently as a gene induced during macrophage colony stimulating factor—initiated maturation of myeloid cells (Kimura et al., 2003, J. Biol. Chem., 278, 25046-25054).

Therefore, in some embodiments of the present invention the “nuclear receptor modulating agent” comprises an isolated full-length HLS-5 polypeptide. The term “polypeptide” refers to a polymer of amino acids and its equivalent and does not refer to a specific length of the product; thus, peptides, oligopeptides and proteins are included within the definition of a polypeptide. This term also does not refer to, or exclude modifications of the polypeptide, for example, glycosylations, acetylations, phosphorylations, and the like. Included within the definition are, for example, polypeptides containing one or more analogs of an amino acid (including, for example, natural amino acids, etc.), polypeptides with substituted linkages as well as other modifications known in the art, both naturally and non-naturally occurring.

Full length HLS-5 polypeptides of the present invention have about 500 amino acids, encode a tumor suppressor factor in an animal, particularly a mammal, and include allelic variants or homologues. Full length HLS-5 polypeptides also typically comprise a Ring finger motif, a B box, a coiled-coil motif and an SPRY motif. HLS-5 polypeptides of the invention also include fragments and derivatives of full length HLS-5 polypeptides, particularly fragments or derivatives having substantially the same biological activity. The polypeptides can be prepared by recombinant or chemical synthetic methods. In some embodiments, the HLS-5 polypeptides include those comprising the amino acid sequence of SEQ ID NO:2, SEQ ID NO:4 or SEQ ID NO:6, or allelic variants or homologues, including fragments, thereof. In further embodiments, the HLS-5 polypeptides consist essentially of amino acids 12 to 504 of the amino acid sequence shown as SEQ ID NO:4 or allelic variants, homologues or fragments, thereof.

In the context of the present invention, a homologous sequence is taken to include an amino acid sequence which is at least 60, 70, 80 or 90% identical, preferably at least 95 or 98% identical at the amino acid level over at least 20, 50, 100, 200, 300 or 400 amino acids with the amino acid sequences set out in SEQ ID NO:2, SEQ ID NO:4, SEQ ID NO:6 or SEQ ID NO:8. In particular, homology should typically be considered with respect to those regions of the sequence known to be essential for the function of the protein rather than non-essential neighbouring sequences. Thus, for example, homology comparisons are preferably made over regions corresponding to the Ring finger, B box, coiled coil and/or SPRY domains of the HLS-5 amino acid sequence set out in SEQ ID NO:2, SEQ ID NO:4 or SEQ ID NO:8. The ring finger corresponds to approximately amino acids 36 to 75 of SEQ ID NO:2. The B box corresponds to approximately amino acids 111 to 152 of SEQ ID NO:2. The coiled coil corresponds to approximately amino acids 219 to 266 of SEQ ID NO:2.

The SPRY domain corresponds to approximately amino acids 368 to 507 of SEQ ID NO:2. In some embodiments, polypeptides of the invention comprise a contiguous sequence having greater than 50, 60 or 70% homology, more preferably greater than 80 or 90% homology, to one or more of amino acids 111 to 152, 219 to 266 or 368 to 507 of SEQ ID NO:2 or the corresponding regions of SEQ ID NO:4 or SEQ ID NO:6.

In some embodiments, polypeptides may alternatively or in addition comprise a contiguous sequence having greater than 80 or 90% homology, to amino acids 36 to 75 of SEQ ID NO:2 or the corresponding region of SEQ ID NO:4 or SEQ ID NO:6. Other polypeptides comprise a contiguous sequence having greater than 40, 50, 60, or 70% homology, more preferably greater than 80 or 90% homology to amino acids 1 to 35, 76 to 110, 153 to 218 and/or 267 to 367 of SEQ ID NO:2 or the corresponding regions of SEQ ID NO:4 or SEQ ID NO:6. Although homology can also be considered in terms of similarity (i.e. amino acid residues having similar chemical properties/functions), in the context of the present invention it is preferred to express homology in terms of sequence identity. The terms “substantial homology” or “substantial identity”, when referring to polypeptides, indicate that the polypeptide or protein in question exhibits at least about 70% identity with an entire naturally-occurring protein or a portion thereof, usually at least about 80% identity, and preferably at least about 90 or 95% identity.

Homology comparisons can be conducted by eye, or more usually, with the aid of readily available sequence comparison programs. These commercially available computer programs can calculate percentage homology between two or more sequences.

Percent (%) homology may be calculated over contiguous sequences, i.e. one sequence is aligned with the other sequence and each amino acid in one sequence directly compared with the corresponding amino acid in the other sequence, one residue at a time. This is called an “ungapped” alignment. Typically, such ungapped alignments are performed only over a relative short number of residues (for example less than 50 contiguous amino acids).

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible-reflecting higher relatedness between the two compared sequences-will achieve a higher score than one with many gaps. “Affine gap costs” are typically used that charge a relative high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example, when using the GCG Wisconsin Bestfit package the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, USA; Devereux et al., 1984, Nucleic Acids Research, 12: 387). Examples of other software than can perform sequence comparisons include, but are not limited to, the BLAST package (see, Ausubel et al., supra), FASTA (Atschul et al., 1990, J. Mol. Biol., 403-410) and the GENEWORKS suite of comparison tools. Both BLAST and FASTA are available for offline and online searching (see Ausubel et al., supra, pages 7-58 to 760). However it is preferred to use the GCG Bestfit program.

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

HLS-5 polypeptide homologues include those having the amino acid sequences, wherein one or more of the amino acids are substituted with another amino acid which substitutions do not substantially alter the biological activity of the molecule.

An HLS-5 polypeptide homologue according to the invention preferably has 80% or greater amino acid sequence identity to the human HLS-5 polypeptide amino acid sequence set out in SEQ ID NO:4 or SEQ ID NO:6. Examples of HLS-5 polypeptide homologues within the scope of the invention include the amino acid sequence of SEQ ID NO:4 or SEQ ID NO:6 wherein: (a) one or more aspartic acid residues is substituted with glutamic acid; (b) one or more isoleucine residues is substituted with leucine; (c) one or more glycine or valine residues is substituted with alanine; (d) one or more arginine residues is substituted with histidine; or (e) one or more tyrosine or phenylalanine residues is substituted with tryptophan.

“Protein modifications or functional fragments” are also encompassed by the term “nuclear receptor modulating agent” when it refers to HLS-5 polypeptides. HLS-5 polypeptides or fragments thereof which are substantially homologous to primary structural sequence but which include, e.g., in vivo or in vitro chemical and biochemical modifications or which incorporate unusual amino acids. Such modifications include, for example, acetylation, carboxylation, phosphorylation, glycosylation, sumoylation, ubiquitination, labelling, e.g., with radionucleotides, and various enzymatic modifications, as will be readily appreciated by those well skilled in the art.

A HLS-5 polypeptide “fragment,” “portion” or “segment” is a stretch of amino acid residues of at least about five to seven contiguous amino acids, often at least about seven to nine contiguous amino acids, typically at least about nine to 13 contiguous amino acids and, most preferably, at least about 20 to 30 or more contiguous amino acids, wherein said “fragment,” “portion” or “segment” has substantially similar function to wild type full length HLS-5 polypeptide.

“Substantially similar function” refers to the function of the polypeptide homologue, variant, derivative or fragment of HLS-5 with reference to the wild-type HLS-5 polypeptide. The “fragment,” “portion” or “segment” of HLS-5 should retain is ability to modulate the nuclear receptor i.e. either up-regulate or down-regulate the activity of the nuclear receptor.

In some embodiments, the “fragment,” “portion” or “segment”, will comprise one or more domains that have been identified in other proteins as being important with respect to function. For example, the B30.2/SPRY domain and an additional domain in huTRIM5alpha, comprising the amino-terminal RING and B-box components of the TRIM motif, have been shown to be required for N-MLV restriction activity, while the intervening coiled-coil domain is necessary and sufficient for huTRIM5alpha multimerization. Truncated huTRIM5alpha proteins that lack either or both the N-terminal RING/B-Box or the C-terminal B30.2/SPRY domain form heteromultimers with full-length huTRIM5alpha and are dominant inhibitors of its N-MLV restricting activity, suggesting that homomultimerization of intact huTRIM5alpha monomers is necessary for N-MLV restriction. However, localization in large cytoplasmic bodies is not required for inhibition of N-MLV by huTRIM5alpha or for inhibition of HIV-1 by chimeric or rhTRIM5alpha (Yap et al., 2005, Curr Biol, 15, 73-78). Geminin is a cellular protein that associates with Cdt1 and inhibits Mcm2-7 loading during S phase. It prevents multiple cycles of replication per cell cycle and prevents episome replication. Geminin forms a parallel coiled-coil homodimer with atypical residues in the dimer interface. Point mutations that disrupt the dimerization abolish interaction with Cdt1 and inhibition of replication. This interactions is essential for replication inhibition (Saxena et al., 2004, Mol Cell, 15, 245-258). Therefore, it is highly likely that functional fragments, portions or segments of HLS-5 will have one or more of the regions identified above. Moreover, techniques well known in the art for identifying or testing the activity of these regions can be used to test or identify if the HLS-5 fragment, portion or segment of the present invention is functional.

In addition to the similarity of function, the modified polypeptide may have other useful properties, such as a longer half-life.

In some embodiments, the HLS-5 fragment is an isoform of HLS-5. HLS-5, like other TRIM proteins, are defined by a cluster of three different RBCC or TRIM protein motifs: a RING motif, which is cysteine-rich and binds zinc; one or two so-called B boxes, which also bind zinc; and a coiled-coil domain that is probably involved in the formation of protein complexes. All individual TRIM proteins homo-oligomerization and some might also form alliances with other TRIM proteins (hetero-oligomerization). There are at least 37 TRIM family members in humans (Reymond et al., 2001, Embo J, 20, 2140-2151). Many TRIM family members have alternative splicing, with the best characterised members being TRIM39 (PML) (Duprez et al., 1999, J Cell Sci, 112, 381-393), TRIM18 (MIDI) (Berti et al., 2004, BMC Cell Biol, 5, 9), TRIM32 (LGMD-2H) (Schoser et al., 2005, Ann Neurol, 57, 591-595) and TRIMS (Xu et al., 2003, Exp Cell Res, 288, 84-93). Each of the various TRIM proteins seems to localize to particular compartments within cells, forming discrete structures to which they entice other proteins, with different isoforms potentially attracting different subsets of proteins and with alternate functions (Reymond et al., 2001, supra). Based upon the foregoing, HLS-5 has at least one isoform.

The HLS-5 isoform shown in SEQ ID NO:6 includes an alternate exon in the coding region which results in a frame shift and an early stop codon, compared to HLS-5 shown in SEQ ID NO:4. SEQ ID NO:6 isoform is shorter and has a distinct C-terminus compared to HLS-5 in SEQ ID NO:4.

In certain embodiments, the HLS-5 nuclear receptor modulating agents are peptidyl compounds (including peptidomimetics) of HLS-5 which have been modified such that they resist or are more resistant to proteolytic degradation and the like. These peptidyl compounds might include functional groups, such as in place of the scissile peptide bond, which facilitates inhibition of a serine-, cysteine- or aspartate-type protease, as appropriate. For example, the HLS-5 peptidyl compound can be a peptidyl diketone or a peptidyl keto ester, a peptide haloalkylketone, a peptide sulfonyl fluoride, a peptidyl boronate, a peptide epoxide, a peptidyl diazomethanes, a peptidyl phosphonate, isocoumarins, benzoxazin-4-ones, carbamates, isocyantes, isatoic anhydrides or the like. Such functional groups have been provided in other peptide molecules, and general routes for their synthesis are known. See, for example, Angelastro et al., 1990, J. Med Chem. 33:11-13; Bey et al., EPO 363,284; Bey et al., EPO 364,344; Grubb et al., WO 88/10266; Higuchi et al., EPO 393,457; Ewoldt et al., 1992, Molecular Immunology, 29(6):713-721; Hernandez et al., 1992, Journal of Medicinal Chemistry, 35(6): 1121-1129; Vlasak et al., 1989, J. Virology 63(5):2056-2062; Hudig et al., 1991, J. Immunol., 147(4):1360-1368; Odakc et al., 1991, Biochemistry, 30(8):2217-2227; Vijayalakshmi et al., 1991, Biochemistry, 30(8):2175-2183; Kam et al., 1990, Thrombosis & Haemostasis, 64(1):133-137; Powers et al., 1989, J. Cell Biochem., 39(1):33-46; Powers et al., Proteinase Inhibitors, Barrett et al., Eds., Elsevier, pp. 55-152 (1986); Powers et al., 1990, Biochemistry, 29(12):3108-3118; Oweida et al., 1990, Thrombosis Research, 58(2):391-397; Hudig et al., 1989, Molecular Immunology, 26(8):793-798; Orlowski et al., 1989, Archives of Biochemistry & Biophysics, 269(1):125-136; Zunino et al., 1988, Biochimica et Biophysica Acta., 967(3):331-340; Kam et al., 1988, Biochemistry, 27(7):2547-2557; Parkes et al., 1985, Biochem J., 230:509-516; Green et al., 1981, J. Biol. Chem., 256:1923-1928; Angliker et al., 1987, Biochem. J., 241:871-875; Puri et al., 1989, Arch. Biochem. Biophys. 27:346-358; Hanada et al., Proteinase Inhibitors: Medical and Biological Aspects, Katunuma et al., Eds., Springer-Verlag pp. 25-36 (1983); Kajiwara et al., 1987, Biochem. Int., 15:935-944; Rao et al., 1987, Thromb. Res., 47:635-637; Tsujinaka et al., 1988, Biochem. Biophys. Res. Commun. 153:1201-1208). See also U.S. Pat. No. 4,935,493; U.S. Pat. No. 5,462,928; U.S. Pat. No. 5,543,396; U.S. Pat. No. 5,296,604; and U.S. Pat. No. 6,201,132.

In other embodiments, the HLS-5 polypeptide is a non-peptidyl compound, e.g., which can be identified by such drug screening assays as described herein. These non-peptidyl compounds can be, merely to illustrate, synthetic organics, natural products, nucleic acids or carbohydrates.

Also included are such peptidomimetics as olefins, phosphonates, aza-amino acid analogs and the like.

Also deemed as equivalents are any HLS-5-based compounds which can be hydrolytically converted into any of the aforementioned HLS-5 compounds including boronic acid esters and halides, and carbonyl equivalents including acetals, hemiacetals, ketals, and hemiketals, and cyclic dipeptide analogs.

The present invention also encompasses pharmaceutically acceptable salts of the HLS-5 compounds include the conventional non-toxic salts or quaternary ammonium salts of the compounds, e.g., from non-toxic organic or inorganic acids. For example, such conventional non-toxic salts include those derived from inorganic acids such as hydrochloride, hydrobromic, sulphuric, sulfonic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

The pharmaceutically acceptable salts of the present invention can be synthesized from the HLS-5 compounds which contain a basic or acid moiety by conventional chemical methods. Generally, the salts are prepared by reacting the free base or acid with stoichiometric amounts or with an excess of the desired salt-forming inorganic or organic acid or base in a suitable solvent.

Contemplated equivalents of the HLS-5 compounds described above include compounds which otherwise correspond thereto, and which have the same general properties thereof (e.g. the ability to control sumoylation), wherein one or more simple variations of substituents are made which do not adversely affect the efficacy of the HLS-5 molecule in use in the contemplated methods. In general, the HLS-5 polypeptides of the present invention may be prepared by the methods described below, or by modifications thereof, using readily available starting materials, reagents and conventional synthesis procedures. In these reactions, it is also possible to make use of variants which are in themselves known, but are not mentioned here.

By the terms “amino acid residue” and “peptide residue” is meant an amino acid or peptide molecule without the —OH of its carboxyl group. In general the abbreviations used herein for designating the amino acids and the protective groups are based on recommendations of the IUPAC-IUB Commission on Biochemical Nomenclature (see Biochemistry (1972) 11:1726-1732). For instance Met, Ile, Leu, Ala and Gly represent “residues” of methionine, isoleucine, leucine, alanine and glycine, respectively. By the residue is meant a radical derived from the corresponding α-amino acid by eliminating the OH portion of the carboxyl group and the H portion of the α-amino group. The term “amino acid side chain” is that part of an amino acid exclusive of the —CH(NH₂)COOH portion, as defined by Kopple, 1966, “Peptides and Amino Acids”, WA Benjamin Inc., New York & Amsterdam, pp 2 and 33; examples of such side chains of the common amino acids are —CH₂CH₂SCH₃ (the side chain of methionine), —CH₂(CH₃)—CH₂CH₃ (the side chain of isoleucine), —CH₂CH(CH₃)₂ (the side chain of leucine) or H— (the side chain of glycine).

For the most part, the amino acids used in the application of this invention are those naturally occurring amino acids found in proteins, or the naturally occurring anabolic or catabolic products of such amino acids which contain amino and carboxyl groups.

The term “amino acid residue” further includes analogs, derivatives and congeners of any specific amino acid referred to herein, as well as C-terminal or N-terminal protected amino acid derivatives (eg. modified with an N-terminal or C-terminal protecting group). For example, the present invention contemplates the use of amino acid analogs wherein a side chain is lengthened or shortened while still providing a carboxyl, amino or other reactive precursor functional group for cyclization, as well as amino acid analogs having variant side chains with appropriate, functional groups). For instance, the HLS-5 polypeptide can include an amino acid analog such as, for example, cyanoalanine, canavanine, djenkolic acid, norleucine, 3-phosphoserine, homoserine, dihydroxy-phenylalanine, 5-hydroxytryptophan, 1-methylhistidine, 3-methylhistidine, diaminiopimelic acid, ornithine, or diaminobutyric acid. Other naturally occurring amino acid metabolites or precursors having side chains which are suitable herein will be recognized by those skilled in the art and are included in the scope of the present invention.

Also included are the (D) and (L) stereoisomers of such amino acids when the structure of the amino acid admits of stereoisomeric forms. The configuration of the amino acids and amino acid residues herein are designated by the appropriate symbols (D), (L) or (DL), furthermore when the configuration is not designated the amino acid or residue can have the configuration (D), (L) or (DL). It will be noted that the structure of some of the compounds of this invention includes asymmetric carbon atoms. It is to be understood accordingly that the isomers arising from such asymmetry are included within the scope of this invention. Such isomers can be obtained in substantially pure form by classical separation techniques and by sterically controlled synthesis. For the purposes of this application, unless expressly noted to the contrary, a named amino acid shall be construed to include both the (D) or (L) stereoisomers.

The phrase “protecting group” as used herein means substituents which protect the reactive functional group from undesirable chemical reactions. Examples of such protecting groups include esters of carboxylic acids and boronic acids, ethers of alcohols and acetals and ketals of aldehydes and ketones. For instance, the phrase “N-terminal protecting group” or “amino-protecting group” as used herein refers to various amino-protecting groups which can be employed to protect the N-terminus of an amino acid or peptide against undesirable reactions during synthetic procedures. Examples of suitable groups include acyl protecting groups such as, to illustrate, formyl, dansyl, acetyl, benzoyl, trifluoroacetyl, succinyl and methoxysuccinyl; aromatic urethane protecting groups as, for example, benzyloxycarbonyl (Cbz); and aliphatic urethane protecting groups such as t-butoxycarbonyl (Boc) or 9-Fluorenylmethoxycarbonyl (FMOC).

Certain polypeptides of the present invention may exist in particular geometric or stereoisomeric forms. The present invention contemplates all such forms, including cis- and trans-isomers, R- and S-enantiomers, diastereomers, (D)-isomers, (L)-isomers, the racemic mixtures thereof, and other mixtures thereof, as, falling within the scope of the invention. Additional asymmetric carbon atoms may be present in a substituent such as an alkyl group. All such isomers, as well as mixtures thereof, are intended to be included in this invention.

The similarity of function (activity) of the modified HLS-5 polypeptide may be substantially the same as the activity of the wild type HLS-5 polypeptide. Alternatively, the similarity of function (activity) of the modified polypeptide may be higher than the activity of the wild-type HLS-5 polypeptide. The function/biological activity of homologues, variant, derivatives or fragments relative to wild type may be determined, for example, by means of biological assays. For example, as described supra there are a number of in vitro and in vivo assays that can be utilised to monitor the effects on the HLS-5 modification on the ability to interact with the nuclear receptor.

The modified polypeptide may be synthesised using conventional techniques, or is encoded by a modified nucleic acid and produced using conventional techniques. The modified nucleic acid is prepared by conventional techniques. A nucleic acid with a function substantially similar to the wild-type HLS-5 gene function produces the modified protein described above.

Besides substantially full-length polypeptides, the present invention provides for biologically active fragments of the polypeptides. Biologically active fragments are those polypeptide fragments retaining nuclear receptor modulating activity.

The present invention also provides for fusion polypeptides, comprising HLS-5 polypeptides and fragments. Homologous polypeptides may be fusions between two or more HLS-5 polypeptide sequences or between the sequences of HLS-5 and a related protein. Likewise, heterologous fusions may be constructed which would exhibit a combination of properties or activities of the derivative proteins.

For example, ligand-binding or other domains may be “swapped” between different new fusion polypeptides or fragments. Such homologous orheterologous fusion polypeptides may display, for example, altered strength or specificity of binding. Fusion partners include immunoglobulins, bacterial p galactosidase, trpE, protein A, β-lactamase, alpha amylase, alcohol dehydrogenase and yeast alpha mating factor.

Fusion proteins will typically be made by either recombinant nucleic acid methods, as described below, or may be chemically synthesized.

“Protein purification” refers to various methods for the isolation of the HLS-5 polypeptides from other biological material, such as from cells transformed with recombinant nucleic acids encoding HLS-5, and are well known in the art. For example, such polypeptides may be purified by immuno-affinity chromatography employing, eg., the antibodies provided by the present invention. Various methods of protein purification are well known in the art.

The terms “isolated”, “substantially pure”, and “substantially homogeneous” are used interchangeably to describe a HLS-5 polypeptide that has been separated from components that accompany it in its natural state. A monomeric protein is substantially purified when at least about 60 to 75% of a sample exhibits a single polypeptide sequence. A substantially purified protein will typically comprise about 60 to 90% W/W of a protein sample, more usually about 95%, and preferably will be over about 99% pure. Protein purity or homogeneity may be indicated by a number of means well known in the art, such as polyacrylamide gel electrophoresis of a protein sample, followed by visualizing a single polypeptide band upon staining the gel. For certain purposes, higher resolution may be provided by using HPLC or other means well known in the art which are utilized for application.

A HLS-5 polypeptide is substantially free of naturally associated components when it is separated from the native contaminants that accompany it in its natural state.

Thus, a HLS-5 polypeptide that is chemically synthesised or synthesised in a cellular system different from the cell from which it naturally originates will be substantially free from its naturally associated components. A protein may also be rendered substantially free of naturally associated components by isolation, using protein purification techniques well known in the art

A HLS-5 polypeptide produced as an expression product of an isolated and manipulated genetic sequence is an “isolated polypeptide,” as used herein, even if expressed in a homologous cell type. Synthetically made forms or molecules expressed by heterologous cells are inherently isolated molecules.

In some embodiments of the present invention the terms “HLS-5 protein” or “HLS-5 polypeptide” refers to a protein or polypeptide encoded by a HLS-5 polynucleotide sequence, variants or functional fragments thereof. Also included are HLS-5 polypeptide encoded by DNA that hybridize under high stringency conditions, to HLS-5 encoding polynucleotides and closely related polypeptides retrieved by antisera to the HLS-5 protein(s). Accordingly, in some embodiments, the term “nuclear receptor modulating agent” comprises an HLS-5 polynucleotide molecule that encodes an HLS-5 polypeptide, allelic variant, or analog, including functional fragments, thereof.

Preferred polynucleotide molecules according to the invention include the polynucleotide sequences set out in SEQ ID NO:1 and SEQ ID NO:3 or functional fragments thereof.

A polynucleotide is said to “encode” a polypeptide if, in its native state or when manipulated by methods well known to those skilled in the art, it can be transcribed and/or translated to produce the RNA for and/or the polypeptide or a fragment thereof. The anti-sense strand is the complement of such a nucleic acid, and the encoding sequence can be deduced therefrom.

An “isolated” or “substantially pure” nucleic acid (e.g., RNA, DNA or a mixed polymer) is one which is substantially separated from other cellular components which naturally accompany a native human sequence or protein, e.g., ribosomes, polymerases, many other human genome sequences and proteins. The term embraces a nucleic acid sequence or protein that has been removed from its naturally occurring environment, and includes recombinant or cloned DNA isolates and chemically synthesized analogs or analogs biologically synthesized by heterologous systems.

“HLS-5 gene sequence,” “HLS-5 gene,” “HLS-5 nucleic acids” or “HLS-5 polynucleotide” include coding sequences, intervening sequences and regulatory elements controlling transcription and/or translation. The term “HLS-5 gene sequence” is intended to include all allelic variations of the DNA sequence.

These terms, when applied to a nucleic acid, refer to a nucleic acid that encodes a HLS-5 polypeptide, fragment, homologue or variant, including, e.g., protein fusions or deletions. The nucleic acids of the present invention will possess a sequence that is either derived from, or substantially similar to a natural HLS-5 encoding gene or one having substantial homology with a natural HLS-5 encoding gene or a portion thereof. The coding sequence for murine HLS-5 polypeptide is shown in SEQ ID NO:1, with the amino acid sequence shown in SEQ ID NO:2. The coding sequence for human HLS-5 polypeptide is shown in SEQ ID NO:3 and SEQ ID NO:7, with the amino acid sequence shown in SEQ ID NO:4 and SEQ ID NO:8.

A nucleic acid or fragment thereof is “substantially homologous” (“or substantially similar”) to another if, when optimally aligned (with appropriate nucleotide insertions or deletions) with the other nucleic acid (or its complementary strand), there is nucleotide sequence identity in at least about 60% of the nucleotide bases, usually at least about 70%, more usually at least about 80%, preferably at least about 90%, and more preferably at least about 95-98% of the nucleotide bases.

Alternatively, substantial homology or (identity) exists when a nucleic acid or fragment thereof will hybridise to another nucleic acid (or a complementary strand thereof) under selective hybridisation conditions, to a strand, or to its complement. Selectivity of hybridisation exists when hybridisation that is substantially more selective than total lack of specificity occurs. Typically, selective hybridisation will occur when there is at least about 55% identity over a stretch of at least about 14 nucleotides, preferably at least about 65%, more preferably at least about 75%, and most preferably at least about 90%. The length of homology comparison, as described, may be over longer stretches, and in certain embodiments will often be over a stretch of at least about nine nucleotides, usually at least about 20 nucleotides, more usually at least about 24 nucleotides, typically at least about 28 nucleotides, more typically at least about 32 nucleotides, and preferably at least about 36 or more nucleotides.

Thus, polynucleotides of the invention preferably have at least 75%, more preferably at least 85%, more preferably at least 90% homology to the sequences shown in the sequence listings herein. More preferably there is at least 95%, more preferably at least 98%, homology. Nucleotide homology comparisons may be conducted as described below for polypeptides. A preferred sequence comparison program is the GCG Wisconsin Best fit program described below. The default scoring matrix has a match value of 10 for each identical nucleotide and −9 for each mismatch. The default gap creation penalty is −50 and the default gap extension penalty is −3 for each nucleotide.

In the context of the present invention, a homologous sequence is taken to include a nucleotide sequence which is at least 60, 70, 80 or 90% identical, preferably at least 95 or 98% identical at the amino acid level over at least 20, 50, 100, 200, 300, 500 or 1000 nucleotides with the nucleotides sequences set out in SEQ ID NO:1 or SEQ ID NO:3. In particular, homology should typically be considered with respect to those regions of the sequence that encode contiguous amino acid sequences known to be essential for the function of the protein rather than nonessential neighbouring sequences. Thus, for example, homology comparisons are preferably made over regions corresponding to the Ring finger, B box, coiled coil and/or SPRY domains of the HLS-5 amino acid sequence set out in SEQ ID NO:2, SEQ ID NO:4 or SEQ ID NO:8.

Preferred polypeptides of the invention comprise a contiguous sequence having greater than 50, 60 or 70% homology, more preferably greater than 80, 90, 95 or 97% homology, to one or more of the nucleotides sequences of SEQ ID NO:1 which encode amino acids 111 to 152, 219 to 266 or 368 to 507 of SEQ ID NO:2 or the equivalent nucleotide sequences in SEQ ID NO:3.

Preferred polynucleotides may alternatively or in addition comprise a contiguous sequence having greater than 80, 90, 95 or 97% homology to the sequence of SEQ ID NO: 1 that encodes amino acids 36 to 75 of SEQ ID NO:2 or the corresponding nucleotide sequences of SEQ ID NO:3. Other preferred polynucleotides comprise a contiguous sequence having greater than 40, 50, 60, or 70% homology, more preferably greater than 80, 90, 95 or 97% homology to the sequence of SEQ ID NO:1 that encodes amino acids 1 to 35, 76 to 110, 153 to 218 and/or 267 to 367 of SEQ ID NO:2 or the corresponding nucleotide sequences of SEQ ID NO:3.

Nucleotide sequences are preferably at least 15 nucleotides in length, more preferably at least 20, 30, 40, 50, 100 or 200 nucleotides in length.

Generally, the shorter the length of the polynucleotide, the greater the homology required to obtain selective hybridization. Consequently, where a polynucleotide of the invention consists of less than about 30 nucleotides, it is preferred that the % identity is greater than 75%, preferably greater than 90% or 95% compared with the HLS-5 nucleotide sequences set out in the sequence listings herein.

Conversely, where a polynucleotide of the invention consists of, for example, greater than 50 or 100 nucleotides, the % identity compared with the HLS-5 nucleotide sequences set out in the sequence listings herein may be lower, for example greater than 50%, preferably greater than 60 or 75%.

Nucleic acid hybridisation will be affected by such conditions as salt concentration, temperature, or organic solvents, in addition to the base composition, length of the complementary strands, and the number of nucleotide base mismatches between the hybridizing nucleic acids, as will be readily appreciated by those skilled in the art. Stringent temperature conditions will generally include temperatures in excess of 30° C., typically in excess of 37° C., and preferably in excess of 45° C. Stringent salt conditions will ordinarily be less than 1000 mM, typically less than 500 mM, and preferably less than 200 mM. However, the combination of parameters is much more important than the measure of any single parameter. An example of stringent hybridization conditions is 65° C. and 0.1×SSC (1×SSC=0.15M NaCl, 0.015M sodium citrate pH 7.0).

The “polynucleotide” compositions of this invention include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions.

Such molecules are known in the art and include, for example, those in which peptide linkages substitute for phosphate linkages in the backbone of the molecule. The present invention provides recombinant nucleic acids comprising all or part of the HLS-5 region. The recombinant construct may be capable of replicating autonomously in a host cell. Alternatively, the recombinant construct may become integrated into the chromosomal DNA of the host cell. Such a recombinant polynucleotide comprises a polynucleotide of genomic, cDNA, semi-synthetic, or synthetic origin which, by virtue of its origin or manipulation, 1) is not associated with all or a portion of a polynucleotide with which it is associated in nature; 2) is linked to a polynucleotide other than that to which it is linked in nature; or 3) does not occur in nature.

Therefore, recombinant nucleic acids comprising sequences otherwise not naturally occurring are provided by this invention. Although the wild-type sequence may be employed, it will often be altered, e.g., by deletion, substitution or insertion.

A “recombinant nucleic acid” is a nucleic acid that is not naturally occurring, or which is made by the artificial combination of two otherwise separated segments of sequence. This artificial combination is often accomplished by either chemical syntheses means, or by the artificial manipulation of isolated segments of nucleic acids, by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions. cDNA or genomic libraries of various types may be screened as natural sources of the nucleic acids of the present invention, or such nucleic acids may be provided by amplification of sequences resident in genomic DNA or other natural sources, e.g., by PCR. The choice of cDNA libraries normally corresponds to a tissue source that is abundant in mRNA for the desired proteins. Phage libraries are normally preferred, but other types of libraries may be used. Clones of a library are spread onto plates, transferred to a substrate for screening, denatured and probed for the presence of desired sequences.

The nucleic acid sequences used in this invention will usually comprise at least about five codons (15 nucleotides), more usually at least about 7-15 codons, and most preferably, at least about 35 codons. This number of nucleotides is usually about the minimal length required for a successful HLS-5 fragment that is still capable of nuclear receptor modification as described herein.

Techniques for nucleic acid manipulation are described generally, for example, in Sambrook et al., 1989, supra or Ausubel et al., 1992, Current Protocols in Molecular Biology. Reagents useful in applying such techniques, such as restriction enzymes and the like, are widely known in the art and commercially available from such vendors as New England BioLabs, Boehringer Mannheim, Amersham, Promega Biotec, US Biochemicals, New England Nuclear and a number of other sources. The recombinant nucleic acid sequences used to produce fusion proteins of the present invention may be derived from natural or synthetic sequences. Many natural gene sequences are obtainable from various cDNA or from genomic libraries using appropriate probes. See, GenBank, National Institutes of Health.

As used herein, the term “HLS-5 gene sequence” refers to the double-stranded DNA comprising the gene sequence or region, as well as either of the single-stranded DNAs comprising the gene sequence or region (i.e. either of the coding and non-coding strands).

As used herein, a “portion” of the HLS-5 gene sequence or region is defined as having a minimal size of at least about eight nucleotides, or preferably about 15 nucleotides, or more preferably at least about 25 nucleotides, and may have a minimal size of at least about 40 nucleotides.

HLS-5 polynucleotide or fragments thereof may be obtained via any known molecular technique. PCR is one such technique that may be used to obtain HLS-5 gene sequences. This technique may amplify, for example, DNA or RNA, including messenger RNA, wherein DNA or RNA may be single stranded or double stranded. In the event that RNA is to be used as a template, enzymes, and/or conditions optimal for reverse transcribing the template to DNA would be utilized. In addition, a DNA-RNA hybrid that contains one strand of each may be utilized. A mixture of nucleic acids may also be employed, or the nucleic acids produced in a previous amplification reaction described herein, using the same or different primers may be so utilise.

The specific nucleic acid sequence to be amplified, i.e., the HLS-5 gene sequence, may be a fraction of a larger molecule or can be present initially as a discrete molecule, so that the specific sequence constitutes the entire nucleic acid. It is not necessary that the sequence to be amplified is present initially in a pure form; it may be a minor fraction of a complex mixture, such as contained in whole human DNA.

DNA utilized herein may be extracted from a body sample, such as blood, tissue material and the like by a variety of techniques such as that described by Maniatis et al. 1982, supra. If the extracted sample has not been purified, it may be treated before amplification with an amount of a reagent effective to open the cells, or animal cell membranes of the sample, and to expose and/or separate the strand(s) of the nucleic acid(s). This lysing and nucleic acid denaturing step to expose and separate the strands will allow amplification to occur much more readily.

The deoxyribonucleotide triphosphates dATP, dCTP, dGTP and dTTP are added to the synthesis mixture, either separately or together with the primers; in adequate amounts and the resulting solution is heated to about 90°-100° C. from about 1 to 10 minutes, preferably from 1 to 4 minutes. After this heating period, the solution is allowed to cool, which is preferable for the primer hybridization. To the cooled mixture is added an appropriate agent for effecting the primer extension reaction (called herein “agent for polymerization”), and the reaction is allowed to occur under conditions known in the art. The agent for polymerization may also be added together with the other reagents if it is heat stable. This synthesis (or amplification) reaction may occur at room temperature up to a temperature above which the agent for polymerization no longer functions.

Thus, for example, if DNA polymerase is used as the agent, the temperature is generally no greater than about 40° C. Most conveniently the reaction occurs at room temperature.

The agent for polymerisation may be any compound or system which will function to accomplish the synthesis of primer extension products, including enzymes.

Suitable enzymes for this purpose include, for example, E. coli. DNA polymerase I, Klenow fragment of E. coli DNA polymerase, polymerase muteins, reverse transcriptase, other enzymes, including heat-stable enzymes (i.e., those enzymes which perform primer extension after being subjected to temperatures sufficiently elevated to cause denaturation), such as Tag polymerase. Suitable enzyme will facilitate combination of the nucleotides in the proper manner to form the primer extension products that are complementary to each HLS-5 gene sequence nucleic acid strand. Generally, the synthesis will be initiated at the 3′ end of each primer and proceed in the 5′ direction along the template strand, until synthesis terminates, producing molecules of different lengths.

The newly synthesised HLS-5 strand and its complementary nucleic acid strand will form a double-stranded molecule under hybridizing conditions described above and this hybrid is used in subsequent steps of the process. In the next step, the newly synthesized HLS-5 double-stranded molecule is subjected to denaturing conditions using any of the procedures described above to provide single-stranded molecules.

The steps of denaturing, annealing, and extension product synthesis can be repeated as often as needed to amplify the target polymorphic gene sequence nucleic acid sequence to the extent necessary for detection. The amount of the specific nucleic acid sequence produced will accumulate in an exponential fashion. Amplification is described in “PCR. A Practical Approach”, ILR Press, Eds. McPherson et al., 1992.

Sequences amplified by the methods of the invention can be further evaluated, detected, cloned, sequenced, and the like, either in solution or after binding to a solid support, by any method usually applied to the detection of a specific DNA sequence such as PCR, oligomer restriction (Saiki et al., 1985, Bio/Technology, 3: 1008-1012), allele-specific oligonucleotide (ASO) probe analysis (Conner et al., 1983, Proc. Natl. Acad. Sci. USA., 80: 278), oligonucleotide ligation assays (OLAs) (Landgren et al., 1988, Science, 241: 1007) and the like. Molecular techniques for DNA analysis have been reviewed (Landgren et al., 1988, Science, 242: 229-237).

Methods of obtaining HLS-5 polynucleotides of the present invention include PCR, as described herein and as commonly used by those of ordinary skill in the art. Alternative methods of amplification have been described and can also be employed as long as the HLS-5 gene sequence amplified by PCR using primers of the invention is similarly amplified by the alternative means. Such alternative amplification systems include, but are not limited to self-sustained sequence replication, which begins with a short sequence of RNA of interest and a T7 promoter. Reverse transcriptase copies the RNA into cDNA and degrades the RNA, followed by reverse transcriptase polymerizing a second strand of DNA. Another nucleic acid amplification technique is nucleic acid sequence-based amplification (NASBA) which uses reverse transcription and T7 RNA polymerase and incorporates two primers to target its cycling scheme. NASBA can begin with either DNA or RNA and finish with either, and amplifies to 108 copies within 60 to 90 minutes.

Alternatively, HLS-5 polynucleotides can be amplified by ligation activated transcription (LAT). LAT works from a single-stranded template with a single primer that is partially single-stranded and partially double-stranded. Amplification is initiated by ligating a cDNA to the promoter oligonucleotide and within a few hours, amplification is 108 to 109 fold. The QB replicase system can be utilized by attaching an RNA sequence called MDV-1 to RNA complementary to a DNA sequence of interest. Upon mixing with a sample, the hybrid RNA finds its complement among the specimen's mRNAs and binds, activating the replicase to copy the tag-along sequence of interest. Another nucleic acid amplification technique, ligase chain reaction (LCR), works by using two differently labelled halves of a sequence of interest that are covalently bonded by ligase in the presence of the contiguous sequence in a sample, forming a new target. The repair chain reaction (RCR) nucleic acid amplification technique uses two complementary and target-specific oligonucleotide probe pairs, thermostable polymerase and ligase, and DNA nucleotides to geometrically amplify targeted sequences. A 2-base gap separates the oligonucleotide probe pairs, and the RCR fills and joins the gap, mimicking normal DNA repair. Nucleic acid amplification by strand displacement activation (SDA) utilizes a short primer containing a recognition site for Hinc II with short overhang on the 5′end that binds to target DNA. A DNA polymerase fills in the part of the primer opposite the overhang with sulphur-containing adenine analogs. Hinc II is added but only cuts the unmodified DNA strand. A DNA polymerase that lacks 5′exonuclease activity enters at the site of the nick and begins to polymerize, displacing the initial primer strand downstream and building a new one which serves as more primer. SDA produces greater than 107-fold amplification in 2 hours at 37° C. Unlike PCR and LCR, SDA does not require instrumented temperature cycling. Another amplification system useful in the method of the invention is the QB Replicase System. Although PCR is the preferred method of amplification if the invention, these other methods can also be used to amplify the HLS-5 gene sequence as described in the method of the invention.

Large amounts of the HLS-5 polynucleotides of the present invention may also be produced by replication in a suitable host cell. Natural or synthetic polynucleotide fragments coding for a desired fragment will be incorporated into recombinant polynucleotide constructs, usually DNA constructs, capable of introduction into and replication in a prokaryotic or eukaryotic cell. Usually the polynucleotide constructs will be suitable for replication in a unicellular host, such as yeast or bacteria, but may also be intended for introduction to (with and without integration within the genome) cultured mammalian or plant or other eukaryotic cell lines.

A double-stranded fragment may be obtained from the single-stranded product of chemical synthesis either by synthesizing the complementary strand and annealing the strands together under appropriate conditions or by adding the complementary strand using DNA polymerase with an appropriate primer sequence.

HLS-5 polynucleotides of the invention may be incorporated into a recombinant replicable vector for introduction into a prokaryotic or eukaryotic host. Such vectors may typically comprise a replication system recognized by the host, including the intended polynucleotide fragment encoding the desired polypeptide, and will preferably also include transcription and translational initiation regulatory sequences operably linked to the polypeptide encoding segment. Expression vectors may include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Secretion signals may also be included where appropriate, whether from a native HLS-5 protein or from other receptors or from secreted polypeptides of the same or related species, which allow the protein to cross and/or lodge in cell membranes, and thus attain its functional topology, or be secreted from the cell. Such vectors may be prepared by means of standard recombinant techniques well known in the art and discussed, for example, in Sambrook et al., 1989 supra or Ausubel et al. 1992 supra.

An appropriate promoter and other necessary vector sequences will be selected so as to be functional in the host, and may include, when appropriate, those naturally associated with HLS-5 genes. Examples of workable combinations of cell lines and expression vectors are described in Sambrook et al., 1989 supra or Ausubel et al., 1992. Many useful vectors are known in the art and may be obtained from such vendors as Stratagene, New England Biolabs, Promega, Biotech, and others. Promoters such as the trp, lac and phage promoters, tRNA promoters and glycolytic enzyme promoters may be used in prokaryotic hosts.

Useful yeast promoters include promoter regions for metallothionein, phosphoglycerate kinase or other glycolytic enzymes such as enolase orglyceraldehyde-3-phosphate dehydrogenase, enzymes responsible for maltose and galactose utilization, and others. Vectors and promoters suitable for use in yeast expression are further described in Hitzeman et al., 1983, Science, 219, pages 620-625.

Appropriate non-native mammalian promoters might include the early and late promoters from SV40 or promoters derived from murine Moloney leukemia virus, mouse tumour virus, avian sarcoma viruses, adenovirus 11, bovine papilloma virus or polyoma. In addition, the construct may be joined to an amplifiable gene (e.g., DHFR) so that multiple copies of the gene may be made.

While such expression vectors may replicate autonomously, they may also replicate by being inserted into the genome of the host cell, by methods well known in the art.

Expression and cloning vectors will likely contain a selectable marker, a gene encoding a protein necessary for survival or growth of a host cell transformed with the vector. The presence of this gene ensures growth of only those host cells that express the inserts. Typical selection genes encode proteins that a) confer resistance to antibiotics or other toxic substances, e.g. ampicillin, neomycin, methotrexate, etc.; b) complement auxotrophic deficiencies, or c) supply critical nutrients not available from complex media, e.g., the gene encoding D-alanine racemase for Bacilli. The choice of the proper selectable marker will depend on the host cell, and appropriate markers for different hosts are well known in the art.

The vectors containing the nucleic acids of interest can be transcribed in vitro, and the resulting RNA introduced into the host cell by well-known methods, e.g., by injection, or the vectors can be introduced directly into host cells by methods well known in the art, which vary depending on the type of cellular host, including electroporation; transfection employing calcium chloride, rubidium chloride, calcium phosphate, DEAE-dextran, or other substances; microprojectile bombardment; lipofection; infection (where the vector is an infectious agent, such as a retroviral genome); and other methods. The introduction of the polynucleotides into the host cell by any method known in the art, including, inter alia, those described above, will be referred to herein as “transformation”. The cells into which have been introduced nucleic acids described above are meant to also include the progeny of such cells.

Thus the present invention provides host cells transformed or transfected with a nucleic acid molecule of the invention. Preferred host cells include bacteria, yeast, mammalian cells, plant cells, insect cells, and human cells.

Illustratively, such host cells are selected from the group consisting of E. coli, Pseudomonas, Bacillus, Streptomyces, yeast, CHO, R1.1, B-W, L-M, COS 1, COS 7, BSC1, BSC40, BMT10, and Sf9 cells.

Large quantities of the HLS-5 polypeptides of the present invention may be prepared by expressing the HLS-5 polynucleotides or portions thereof in vectors or other expression vehicles in compatible prokaryotic or eukaryotic host cells. The most commonly used prokaryotic hosts are strains of Escherichia coli, although other prokaryotes, such as Bacillus subtilis or Pseudomonas may also be used.

Also provided are mammalian cells containing an HLS-5 polypeptide encoding DNA sequence and modified in vitro to permit higher expression of HLS-5 polypeptide by means of a homologous recombinational event consisting of inserting an expression regulatory sequence in functional proximity to the HLS-5 polypeptide encoding sequence. The expression regulatory sequence can be an HLS-5 polypeptide expression or not and can replace a mutant HLS-5 polypeptide regulatory sequence in the cell.

Thus, the present invention also provides methods for preparing an HLS-5 polypeptide comprising: (a) culturing a cell as described above under conditions that provide for expression of the HLS-5 polypeptide; and (b) recovering the expressed HLS-5 polypeptide. This procedure can also be accompanied by the steps of: (c) chromatographing the polypeptide using any suitable means known in the art; and (d) purifying the polypeptide by for example gel filtration.

Mammalian or other eukaryotic host cells, such as those of yeast, filamentous fungi, plant, insect, or amphibian or avian species, may also be useful for production of the proteins of the present invention. Propagation of mammalian cells in culture is per se well known. Examples of commonly used mammalian host cell lines are VERO and HeLa cells, Chinese hamster ovary (CHO) cells, and W138, BHK, and COS cell lines, although it will be appreciated by the skilled practitioner that other cell lines may be appropriate, e.g., to provide higher expression, desirable glycosylation patterns, or other features.

Clones are selected by using markers depending on the mode of the vector construction. The marker may be on the same or a different DNA molecule, preferably the same DNA molecule. In prokaryotic hosts, the transformant may be selected, e.g., by resistance to ampicillin, tetracycline or other antibiotics.

Production of a particular product based on temperature sensitivity may also serve as an appropriate marker.

Prokaryotic or eukaryotic cells transformed with the polynucleotides of the present invention will be useful not only for the production of the nucleic acids and polypeptides of the present invention.

It will be appreciate by those skilled in that that an increase in the amount intracellular amount of HLS-5 can be obtained either by the techniques described supra or by increasing the endogenous levels of HLS-5. Accordingly, in some embodiments the “nuclear receptor modulating agent” is a compound or composition capable of altering the endogenous levels of HLS-5 and/or HLS-5 activity. The term “altering” as used herein with reference to the endogenous levels of HLS-5 refers to the ability of the compound or composition to increase or decrease the endogenous levels of HLS-5 and/or HLS-5 activity as compared to the wild-type and/or normal levels. Compounds and/or composition capable of modifying the endogenous levels of HLS-5 and/or HLS-5 activity can be initially identified using in vitro cell based assays. For example, a system such as Chroma-Luc™, Luc' or GFP™ reporter genes can be provided in multiple different cloning vector formats. The Basic vector versions are general-purpose reporter vectors based on the design, for example of the pGL3-Basic Vector, which lacks eukaryotic promoter and enhancer sequences, allowing cloning putative regulatory sequences, such as the HLS-5 promoter at the 5′ end of the reporter gene. Expression of luciferase, or any reporter gene, activity in cells transfected with this “pGL3-Promoter Vector” depends on elements or compounds being able to induce directly or indirectly the expression through the cloned promoter of interest, such as the HLS-5 promoter. In addition to the basic vector configuration, other systems such as the Chroma-Luc™ genes are available in a vector configuration containing an SV40 promoter and SV40 enhancer, similar to the pGL3-Control Vector. The presence of the SV40 promoter and enhancer sequences result in strong expression of luc+ in many types of mammalian cells. Thus this technology and any other vector modification is suitable for rapid quantitation in multiwell plates and in high-throughput applications to assay for compounds which are potentially capable of modifying the HLS-5 protein expression by measuring the reporter gene downstream of the HLS-5 promoter. These identified compounds can than be tested in cells with the endogenous HLS-5 promoter and protein expression assayed by such methods as Western Blots. In general, any luminometer capable of measuring filtered luminescence should be able to perform dual-colour assays and any scientist skilled in the art can reproduce these assays.

Once the nuclear receptor modulating agents eg HLS-5 polypeptide, HLS-5 polynucleotide in appropriate vector or compound/composition capable of altering the endogenous levels of HLS-5 and/or HLS-5 activity, have been obtained they are then administered to a subject in need thereof in order to modulate the nuclear receptors. In some embodiments, the subject in need is suffering from a condition, which is affected, by, controlled by or exacerbated by unregulated nuclear receptor activity and therefore, the step of administration assists in the treatment of the condition.

Generally, the terms “treating,” “treatment” and the like are used herein to mean affecting a subject e.g. human individual or animal, their tissue or cells to obtain a desired pharmacological and/or physiological effect. The effect may be prophylactic in terms of completely or partially preventing the condition or sign or symptom thereof, and/or may be therapeutic in terms of a partial or complete cure of the condition. “Treating” as used herein covers any treatment of, or prevention of a condition associated with or exacerbated by unregulated nuclear receptor activity in a vertebrate, a mammal, particularly a human, and includes: (a) preventing the condition from occurring in a subject that may be predisposed to the condition, but has not yet been diagnosed as having it; (b) inhibiting the condition, i.e., arresting its development; or (c) relieving or ameliorating the condition, i.e., cause regression of the symptoms.

The term “subject” as used herein refers to an animal subject in which the control of nuclear receptor activity is desirable. The subject may be a human, or may be a domestic, companion or zoo animal. While it is particularly contemplated that the nuclear receptor modulating agent of the invention is suitable for use in medical treatment of humans, it is also applicable to veterinary treatment, including treatment of companion animals such as dogs and cats, and domestic animals such as horses, cattle and sheep, or zoo animals such as non-human primates, felids, canids, bovids, and ungulates.

The nuclear receptor modulating agents can be administered in various forms, depending on the condition to be treated and the age, condition and body weight of the subject, as is well known in the art. For example, where the nuclear receptor modulating agents are to be administered orally, they may be formulated as tablets, capsules, granules, powders or syrups; or for parenteral administration, they may be formulated as injections (intravenous, intramuscular or subcutaneous), drop infusion preparations or suppositories. For application by the ophthalmic mucous membrane route, they may be formulated as eye drops or eye ointments. These formulations can be prepared by conventional means, and, if desired, the active ingredient may be mixed with any conventional additive, such as an excipient, a binder, a disintegrating agent, a lubricant, a corrigent, a solubilizing agent, a suspension aid, an emulsifying agent or a coating agent. Although the dosage will vary depending on the symptoms, age and body weight of the subject, the nature and severity of the condition to be treated or prevented, the route of administration and the form of the nuclear receptor modulating agent, in general, a daily dosage of from 0.01 to 2000 mg of the nuclear receptor modulating agents is recommended for an adult human subject, and this may be administered in a single dose or in divided doses.

An effective time for administering the nuclear receptor modulating agent needs to be identified. This can be accomplished by routine experiments. For example, in animals, the control of sumoylation activity by the nuclear receptor modulating agent can be assessed by administering the nuclear receptor modulating agent at a particular time of day and measuring the effect of the administration (if any) by measuring one or more indices associated with nuclear receptor activity, and comparing the post-treatment values of these indices to the values of the same indices prior to treatment.

The precise time of administration and/or amount of nuclear receptor modulating agent that will yield the most effective results in terms of efficacy of treatment in a given subject will depend upon the activity, pharmacokinetics, and bioavailability of a particular nuclear receptor modulating agent, physiological condition of the subject (including age, sex, disease type and stage, general physical condition, responsiveness to a given dosage and type of medication), route of administration, etc. However, the above guidelines can be used as the basis for fine-tuning the treatment, eg., determining the optimum time and/or amount of administration, which will require no more than routine experimentation consisting of monitoring the subject and adjusting the dosage and/or timing.

The phrases “pharmaceutically-effective amount” and “therapeutically-effective amount” as used herein means that amount of a nuclear receptor modulating agent, which is effective for producing some desired therapeutic effect, for example, the inhibition of sumoylation of a protein at a reasonable benefit/risk ratio applicable to any medical treatment.

The phrase “pharmaceutically acceptable” is employed herein to refer to those nuclear receptor modulating agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the nuclear receptor modulating agents from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid, (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as colouring agents, release agents, coating agents, sweetening, flavouring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin; propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations useful in the methods of the present invention include those suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a nuclear receptor modulating agent(s) with the carrier and, optionally, one or more accessory ingredients. In general, the formulations are prepared by uniformly and intimately bringing into association a nuclear receptor modulating agent with liquid carriers, or finely divided solid carriers, or both, and then, if necessary, shaping the product.

Formulations suitable for oral administration may be in the form of capsules, cachets, pills, tablets, lozenges (using a flavoured basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatine and glycerine, or sucrose and acacia) and/or as mouth washes and the like, each containing a predetermined amount of a nuclear receptor modulating agent(s) as an active ingredient. A compound may also be administered as a bolus, electuary or paste.

In solid dosage forms for oral administration (capsules, tablets, pills, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) colouring agents. In the case of capsules, tablets and pills, the pharmaceutical compositions may also comprise buffering agents. Solid compositions of a similar type, may also be employed as fillers in soft and hard-filled gelatine capsules using such excipients as lactose or milk sugars, as well as high molecular weight polyethylene glycols and the like.

A tablet may be made by compression or moulding, optionally with one or more accessory ingredients. Compressed tablets may be prepared using binder (for example, gelatine or hydroxypropylmethyl cellulose), lubricant, inert diluent, preservative, disintegrant (for example, sodium starch glycolate or cross-linked sodium carboxymethyl cellulose), surface-active or dispersing agent. Moulded tablets may be made by moulding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isodropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavouring, colouring, perfuming and preservative agents

Suspensions, in addition to the active nuclear receptor modulating agent(s) may contain suspending agents as, for example, ethoxylated isostearyl alcohols, polyoxyethylene sorbitol and sorbitan esters, microcrystalline cellulose, aluminium metahydroxide, bentonite, agar-agar and tragacanth, and mixtures thereof.

Dosage forms for the topical or transdermal administration of a nuclear receptor modulating agent(s) include powders, sprays, ointments, pastes, creams, lotions, gels, solutions, patches and inhalants. The active component may be mixed under sterile conditions with a pharmaceutically-acceptable carrier, and with any preservatives, buffers, or propellants which may be required.

The ointments, pastes, creams and gels may contain, in addition to nuclear receptor modulating agent(s), excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a nuclear receptor modulating agent(s), excipients such as lactose, talc, silicic acid, aluminium hydroxide, calcium silicates and polyamide powder, or mixtures of these substances. Sprays can additionally contain customary propellants, such as chlorofluorohydrocarbons and volatile unsubstituted hydrocarbons, such as butane and propane.

The nuclear receptor modulating agent(s) can be alternatively administered by aerosol. This is accomplished by preparing an aqueous aerosol, liposomal preparation or solid particles containing the compound. A non-aqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically acceptable carriers and stabilizers. The carriers and stabilizes vary with the requirements of the particular compound, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a nuclear receptor modulating agent(s) to the body. Such dosage forms can be made by dissolving or dispersing the agent in the proper medium. Absorption enhancers can also be used to increase the flux of the peptidomimetic across the skin. The rate of such flux can be controlled by either providing a rate controlling membrane or dispersing the peptidomimetic in a polymer matrix or gel.

Ophthalmic formulations, eye ointments, powders, solutions and the like, are also contemplated as being within the scope of this invention.

Pharmaceutical compositions of this invention suitable for parenteral administration comprise one or more nuclear receptor modulating agent(s) in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various anti-bacterial and anti-fungal agents, for example, paraben, chlorobutanol, phenol sorbic-acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminium monostearate and gelatine.

In some cases, in order to prolong the effect of a nuclear receptor modulating agent, it is desirable to slow the absorption of the agent from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered agent form is accomplished by dissolving or suspending the agent in an oil vehicle.

Injectable depot forms are made by forming microencapsule matrices of nuclear receptor modulating agent(s) in biodegradable polymers such as polylactide-polyglycolide. Depending on the ratio of agent to polymer, and the nature of the particular polymer employed, the rate of agent release can be controlled. Examples of other biodegradable polymers include poly(orthoesters) and poly(anhydrides). Depot injectable formulations are also prepared by entrapping the agent in liposomes or microemulsions which are compatible with body tissue.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a nuclear receptor modulating agent other than directly into the central nervous system, such that it enters the subjects system and, thus, is subject to metabolism and other like processes, for example, subcutaneous administration.

Another aspect of the invention provides a conjoint therapy wherein one or more other therapeutic agents are administered with the nuclear receptor modulating agent. Such conjoint treatment may be achieved by way of the simultaneous, sequential or separate dosing of the individual components of the treatment.

In some embodiments, a nuclear receptor modulating agent is conjointly administered with anti-cancer agents or other therapeutic agents known to be useful in the treatment of the condition being treated. For example, gene therapy using HLS-5 expression vector together with an anti-cancer agent.

In another illustrative embodiment, the subject control agents can be conjointly administered with a nuclear receptor agonist or antagonist.

As described supra, in some embodiments the HLS-5 polynucleotides and vectors of the invention for in vivo delivery and expression. This approach has also been called “gene therapy” and as such is well known in the art. Gene therapy protocols may involve administering an therapeutically-effective amount of a HLS-5 polynucleotide vector capable of directing expression of the HLS-5 polypeptide to a subject either before, substantially contemporaneously, with, or after influenza virus infection. Another approach that may be used alternatively or in combination with the foregoing is to isolate a population of cells, e.g., stem cells or immune system cells from a subject, optionally expand the cells in tissue culture, and administer a HLS-5 polynucleotide vector capable of directing expression of HLS-5 to the cells in vitro. The cells may then be returned to the subject. Optionally, cells expressing the HLS-5 polynucleotides can be selected in vitro prior to introducing them into the subject. In some embodiments of the invention a population of cells, which may be cells from a cell line or from an individual who is not the subject, can be used. Methods of isolating stem cells, immune system cells, etc., from a subject and returning them to the subject are well known in the art. Such methods are used, eg., for bone marrow transplant, peripheral blood stem cell transplant, etc., in patients undergoing chemotherapy.

In yet another approach, oral gene therapy may be used. For example, U.S. Pat. No. 6,248,720 describes methods and compositions whereby genes under the control of promoters are protectively contained in microparticles and delivered to cells in operative form, thereby achieving non-invasive gene delivery. Following oral administration of the microparticles, the genes are taken up into the epithelial cells, including absorptive intestinal epithelial cells, taken up into gut associated lymphoid tissue, and even transported to cells remote from the mucosal epithelium. As described therein, the microparticles can deliver the genes to sites remote from the mucosal epithelium, i.e. can cross the epithelial barrier and enter into general circulation, thereby transfecting cells at other locations.

The term “condition” refers to any disease or disorder in which nuclear receptor activity plays apart and requires controlling. At present there are a number of conditions known to be affected by unregulated nuclear receptor activity including, but not limited to, androgen-dependent and independent prostate cancer, breast cancer, osteoporosis, Alzheimer's, cerebrovascular disease, pre-eclampsia and endometriosis.

The present invention also provides assays that are suitable for identifying substances that bind to HLS-5 polypeptides (reference to which includes homologues, variants, derivatives and fragments as described above). In addition, assays are provided that are suitable for identifying substances that interfere with HLS-5 binding to cellular components involved in nuclear receptor activity, for example proteins identified in yeast two-hybrid screens as interacting with HLS-5. Such assays are typically in vitro. Assays are also provided that test the effects of candidate substances identified in preliminary in vitro assays on intact cells in whole cell assays.

A substance that modifies nuclear receptor activity as a result of an interaction with HLS-5 polypeptides may do so in several ways. It may directly disrupt the binding of HLS-5 to a cellular component of the cell cycle machinery by, for example, binding to HLS-5 and masking or altering the site of interaction with the other component. Candidate substances of this type may conveniently be preliminarily screened by in vitro binding assays as, for example, described above and then tested, for example in a whole cell assay as described supra.

By “comprising” is meant including, but not limited to, whatever follows the word comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The following examples, which describe exemplary techniques and experimental results, are provided for the purpose of illustrating the invention, and should not be construed as limiting.

### Example 1

### HLS-5 Gene Mutated in Human Cancers Cell Lines

To determine if the HLS-5 gene was mutated in human cancers, DNA from a number of cell lines as well as primary tumours was examined. The cell lines analysed by direct DNA sequencing were 21-MT, MCF7, T47D and LNCAP—these are breast and prostate cancer lines with 3 of them displaying loss of the 8p21 region. In addition, 25 primary breast tumours and 10 breast cell lines were analysed by dHPLC. However, in all cases, no mutations to the hHLS5v1 Open Reading Frame were detected. Thus, so far, it appears less likely that mutations to HLS-5 are responsible for its contribution to cancer.

To demonstrate whether HLS-5 levels were reduced in human cancers, a quantitative polymerase chain reaction (qPCR) assay was developed using primers around the BBox region:

| | |
|---|---|
| 5′-TTCGGCTCTTGTGTTTCATGAG-3′ | SEQ ID NO: 9 |
| 3′-TCAGGTGGAGGCTGCATG-5′ | SEQ ID NO: 10 |

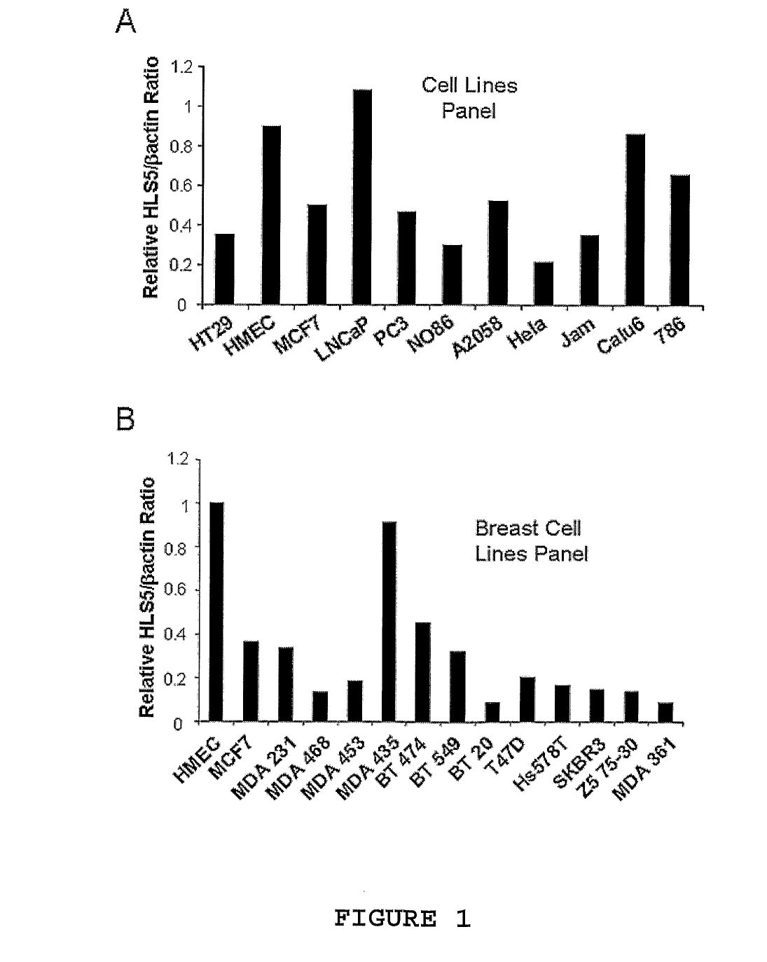

A panel of cell lines representing a wide variety of human tumours was then analysed by qPCR. FIG. 1 Panel (A) shows the levels of HLS5 mRNA as measured by real time RT-PCR in eleven different cancer cell lines: Colon (HT-29), breast (MCF7), prostate (LNCaP, PC3), NO86 (mesothelioma), A2058 (metastatic melanoma), HeLa (cervical carcinoma), JAM (ovarian carcinoma), Calu6 (Non-Small Cell Lung Cancer), 786-0 (Renal Carcinomas). Panel (B) shows the normal human mammary epithelial cells (HMEC) and the remaining are breast cancer cell lines. Importantly, the data showed that 10/13 breast cancer lines had significantly lower HLS-5 transcript levels than control cells. In addition, HLS-5 expression was low in 1/2 prostate cancer lines, 1/1 mesothelioma, 1/1 cervical cancer and 1/1 ovarian cancers.

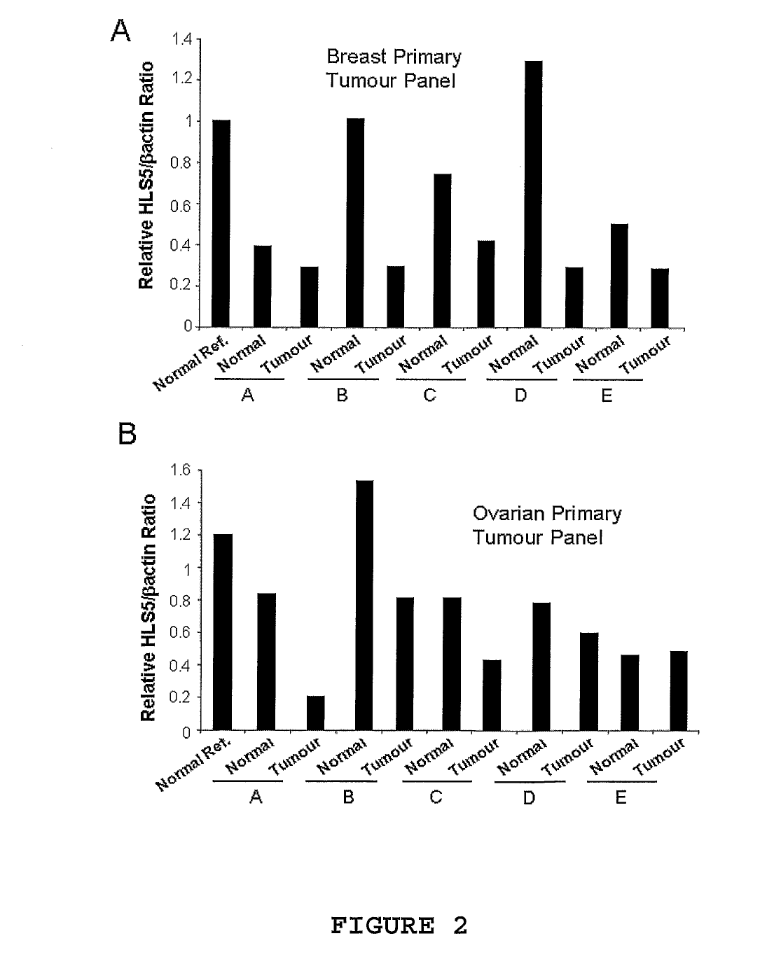

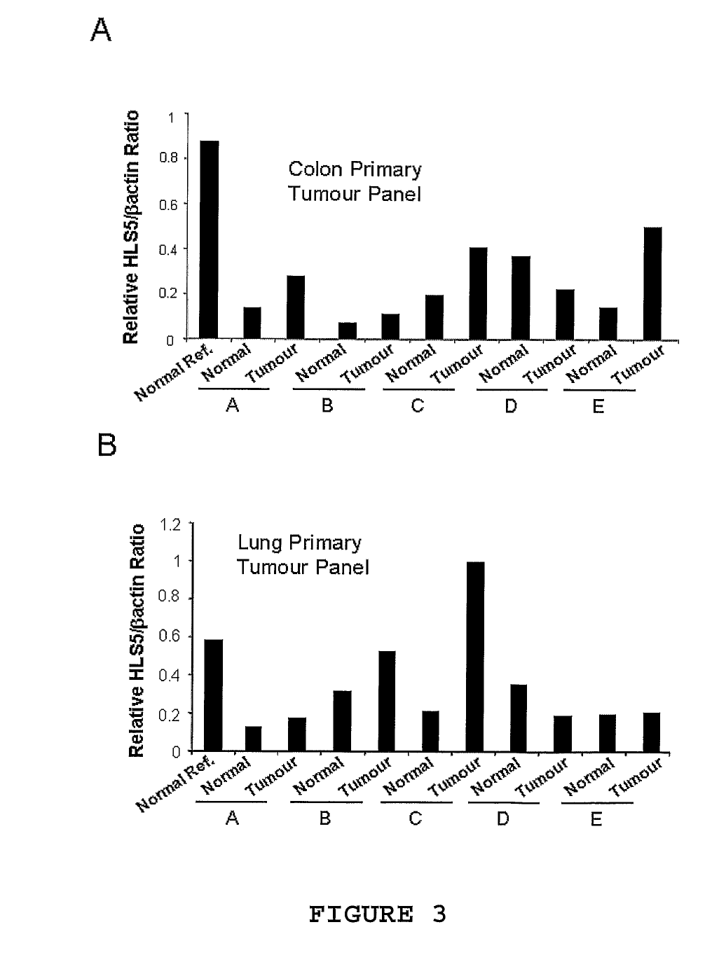

To expand on these observations RNA was obtained from primary breast, lung, ovary and liver cancers, as well as several leukemias. Significantly, HLS5 mRNA levels are widely suppressed in primary breast and ovarian cancers (FIG. 2). Individual tumours with their normal surrounding tissues have been labelled from A to E. However, as shown in FIG. 3 HLS5 mRNA is partially induced in primary colon and lung cancers. Individual tumours with their normal surrounding tissues have been labelled from A to E.

Taken together, the data from the cell lines and primary tumours indicated that HLS-5 levels were reduced noticeably in breast, ovarian, uterine and prostate cancers. These tumours are classified as hormone-dependent cancers and have a mechanism of action through the androgen and oestrogen receptors.

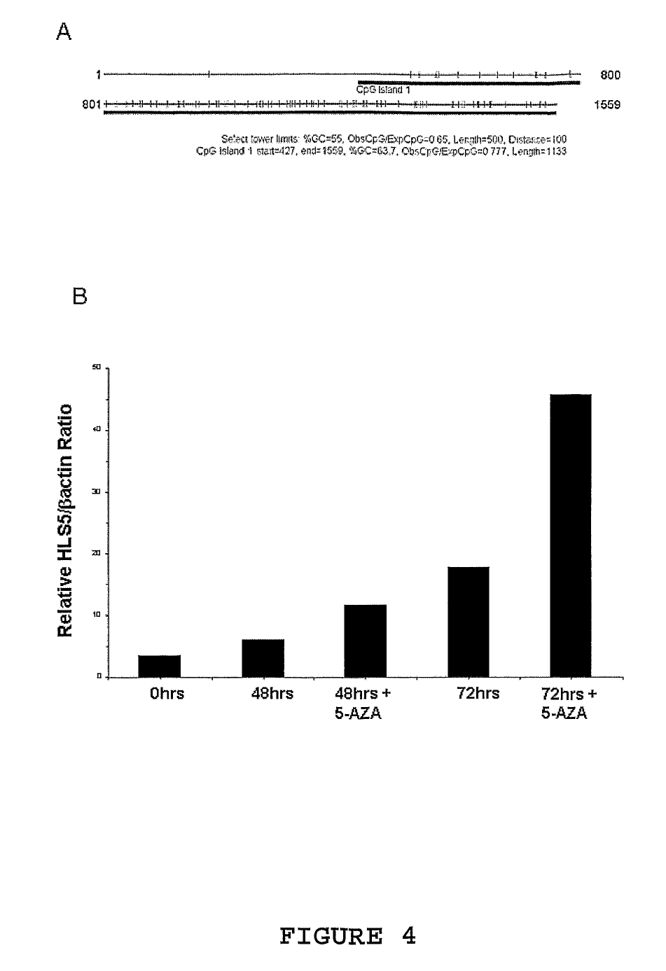

Because of the decreased likelihood of mutations in hHLS-5 mentioned above, it was hypothesized that the reduction in HLS-5 transcripts in these human cancers was due to methylation. Since methylation of genes occurs primarily in CpG islands, an examination of the human HLS-5 genomic structure was conducted to identify possible CpG islands. FIG. 4 shows analysis of possible epigenetic modification of HLS5. Panel (A) shows the region within the HLS5 promoter and first exon which is likely to be a CpG island using the The CpG Island Searcher (http://www.bioinfo.de/isb/2003/03/0021/main.html). Panel (B) shows the 5-azacytidine-induced re-expression of HLS5 in a MDA-435 cell line.

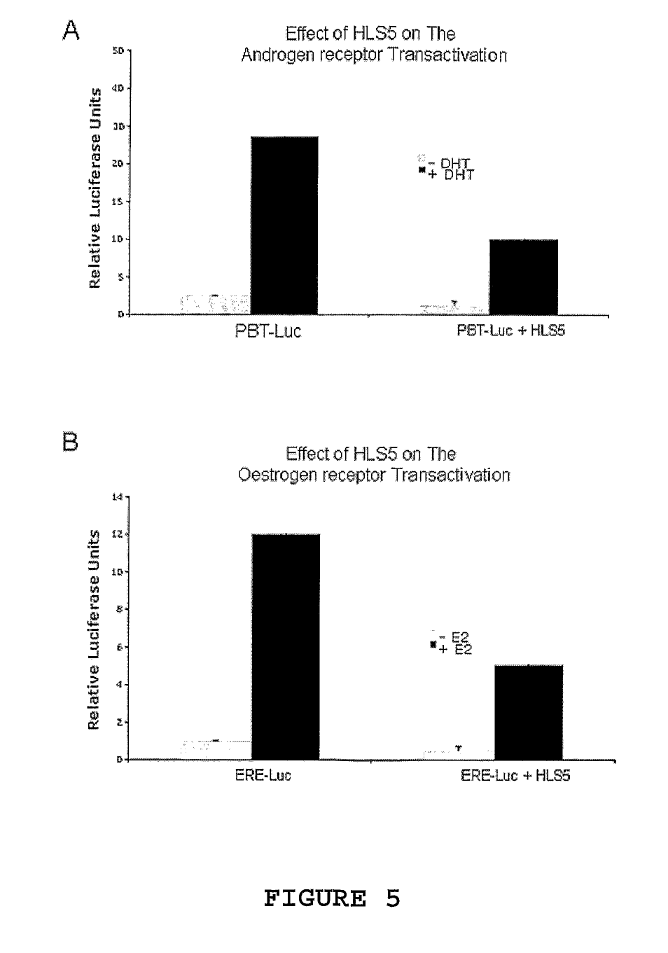

In addition, these cell lines were treated with 5-azacytidine, a demethylating agent. It was hypothesized that the low expressing breast cancer cell line would express higher levels of HLS-5 following removal of the inhibitory methyl groups. FIG. 5 Panel (A) shows HLS5 expression vector pcDNA3-HLS5 (0.5 μg) and reporter vector pBT-Luciferase (1 μg) were co-transfected into LNCaP cells in 6-cm dishes, and the effect of HLS5 on AR transactivation was tested in combination with dihydrotestosterone (DHT). HeLa cells (7.5×10⁴) were plated in 6-well plates 24 h prior transfection. Transfections were performed using Lipofectamine in accordance with the manufacturers instructions. The transfected DNA included 500 ng of reporter plasmid (ERE-Luc) and 25 ng of pRL-CMV Renilla luciferase vector (Promega) used as internal control, together with various amounts of expression vectors, as indicated. Total transfected DNA was kept constant by adding empty pSG5-Flag vectors or pcDNA3. Cells were incubated in the absence or presence of 0.1 nM dihydrotestosterone for 24 h following transfection, then harvested after an additional 24 h and assayed for luciferase activity following the manufacturer's instructions. Luciferase activities were normalized to the activity of the internal control Renilla luciferase and to the protein amounts in samples. Each set of experiments was performed in triplicate and repeated at least three times.

Vector alone did not inhibit AR transactivation. FIG. 5 Panel (B) shows the HLS5 expression vector pcDNA3-HLS5 (0.5 μg) and reporter vector ERE-Luciferase (1 μg) were co-transfected into MCF-7 cells in 6-cm dishes, and the effect of HLS5 on ER transactivation was tested in combination with oestradiol (E2). Cells were incubated in the absence or presence of 0.1 nM E2. DNA was balanced with equimolar amounts of pcDNA3 empty vector or pcDNA3-HLS5. Vector alone did not inhibit ER transactivation.

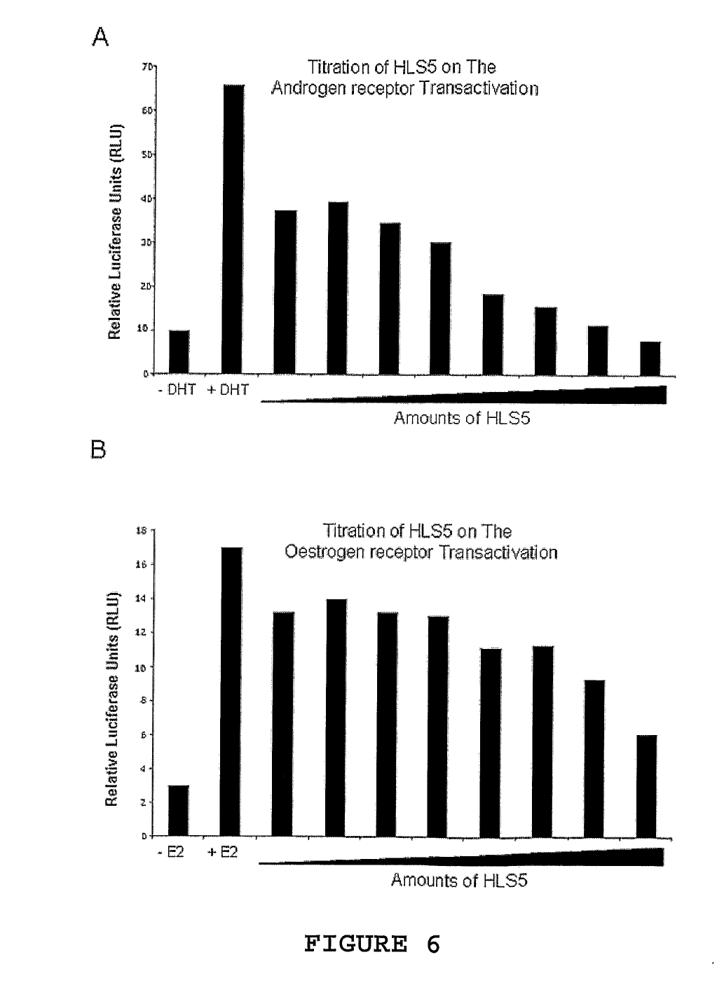

Hormone-dependent cancers require activation of steroid hormone receptors by steroid hormones (eg oestrogen, androgen) for the development of these neoplasms. To determine whether HLS-5 could inhibit steroid hormone receptors, luciferase assays were utilized to measure transcriptional activity of the receptors. Strikingly, FIG. 6 shows that HLS5 inhibited the transcriptional activity by the Andogen and oestrogen receptor in a dose-dependent manner. Panel (A) Assays were performed as in FIG. 5 with transfections of increasing amounts of pcDNA3-HLS5-Myc expression vector DNA (indicated by numbers 3-10, from 25 ng to 5 μg); (controls 1-2), pcDNA3+PBT-luciferase alone or in the presence of dihydrotestosterone; The graphs represent activity measured in the presence of 0.1 nM dihydrotestosterone. Panel (B) shows that assays performed as in FIG. 5 with transfections of increasing amounts of pcDNA3-HLS5 expression vector DNA (indicated by numbers 3-10); (controls 1-2), pcDNA3+ERE-luciferase alone or in the presence of oestradiol; The graphs represent activity measured in the presence of 0.1 nM oestradiol. Collectively, these data demonstrate that HLS-5 is capable of reducing steroid hormone receptor activity. Therefore, reduction of HLS-5 in hormone-dependent cancers removes a key negative regulatory element of steroid hormone action.

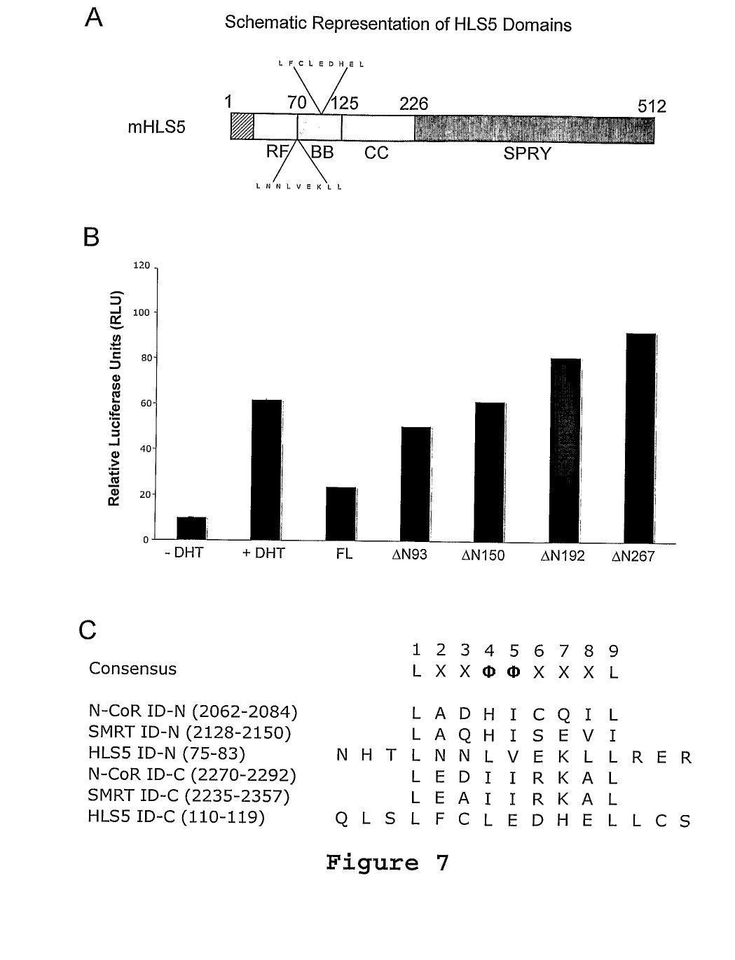

Domain mapping of the HLS-5 molecule was undertaken to define regions which may be responsible for the inhibition of steroid hormone receptor activity. FIG. 7 demonstrated that the central portion of the HLS-5 protein, encompassing the B Box & coiled-coil domain was primary involved in the suppression of receptor transactivation. Panel (A) is a schematic representation of HLS5 Amino Acid sequence. The following domains are highlighted: Ring Finger (RF), B Box (BB), Coiled Coil Domain (CC), and the SPRY Domain. Panel (B) Mutational analysis of HLS5 for inhibition of transcriptional activity on the androgen receptor shows that transfection of deletion variants of HLS5 (ΔN93) still had transcriptional repression compared to the full length HLsS. The remaining variants, (ΔN150, ΔN192, ΔN267) encoded an HLS5 protein with no repression activity. Panel (C) shows the identification of the nuclear receptor co-repressors present on HLS5, N-CoR and SMRT which contain multiple repressor domains that could transfer their active repression function, recruiting histone deacetylases (HDACs). Sequences referred to as the CoRNR box [1] or alternatively as LXXΦΦXXX I/L motifs [2], were is a hydrophobic residue, appear to bind in the hydrophobic pocket that is occupied by the co-activator LXXLL helical motifs upon binding of ligand.

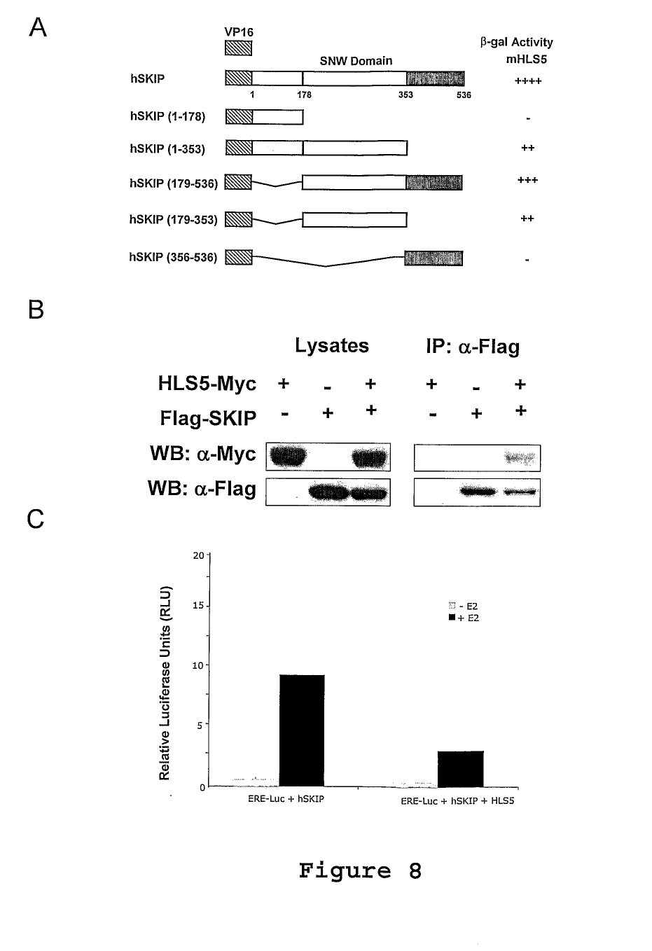

Androgen receptor activity is enhanced by several molecules including Ubc9 and PIAS1. These 2 proteins were shown to interact with HLS-5 in a yeast 2 hybrid screen. The interaction between HLS-5 and Ubc9, as well as PIAS1, has been confirmed biochemically. Importantly, the enhanced activation of steroid hormone receptors by Ubc9 or PIAS1 were markedly suppressed by HLS-5. FIG. 8 shows the identification of minimal receptor interaction domains between hSKIP and in HLS5. Panel (A) shows a schematic representation of hSKIP domain structure (sequence numbering corresponds to full-length Human Skip). The interaction of the different VP16-hSKIP fusion constructs with HLS5 was evaluated using the Yeast two-hybrid System and shown to be mediated through the SNW domain. This SNW domain, which is essential for its function as also been found to be critical for SKIP interaction with the oncoproteins Ski and E6 (Prathapam, T. Oncogene 20:7677, 2001 and Prathapam T. Nuc Acid Research 29:3469, 2001). Panel (B) shows co-immunoprecipitation between hSKIP and HLS5 in Cos cells using HA tagged SKIP and Myc Tagged HLS5. Panel (C) shows HLS5 expression repressed the SKIP induced transactivation on the oestrogen response element ERE. The HLS5 expression vector pcDNA3-HLS5 (0.5 μg) and reporter vector ERE-Luciferase (1 μg) were co-transfected into MCF-7 cells in 6-cm dishes, and the effect of SKIP+/−HLS5 on ER transactivation was tested in combination with Oestradiol (E2). Cells were also incubated in the absence or presence of 0.1 nM E2. DNA was balanced with equimolar amounts of pcDNA3 empty vector or pcDNA3-HLS5. Vector alone did not inhibit ER transactivation. These data add weight to the proposition that HLS-5 is a crucial negative regulator of steroid hormone receptors.

A yeast 2 hybrid screen using the Full length HLS-5 also identified 4 other molecules which associate with steroid hormone receptors viz Rho-GDI, BOAS 2, NRBF-2, SKIP and TDG, interacting with HLS-5. Here we show also evidence of HLS-5 acting as a co-repressor through at least 2 of these activators.

SKIP, which encodes the nuclear Ski interacting protein (Skip). Skip is the human homolog of Drosophila melanogaster nuclear protein Bx42 (Dahl et al., 1998, Oncogene, 16, 1579-1586). Recent evidence suggests that SkiP, like its Drosophila counterpart, plays an important role in transcriptional regulation and is widely expressed in many tissues. It was first described as a nuclear protein that interacted with the oncoprotein v-Ski to mediate its transforming activities (Dahl et al., 1998, supra). Recent studies demonstrate that SkiP interacts with many transcriptional regulators to modulate transcription. Some of the SkiP partners recently identified include NotchIC (Zhou et al., 2000, Mol Cell Biol, 20, 2400-2410), Smad proteins (Leong et al., 2001, J Biol Chem, 276, 18243-18248), retinoblastoma tumor suppressor (Prathapam et al., 2002, Nucleic Acids Res, 30, 5261-5268), vitamin D receptor (VDR) (Zhang et al., 2001, J Biol Chem, 276, 40614-40620), N-CoR/SMRT, and p300 (Leong et al., 2004, Biochem Biophys Res Commun, 315, 1070-1076). Increased SKIP expression has been found to enhance gene expression mediated by vitamin D, retinoic acid, estrogen, and glucocorticoid (Baudino et al., 1998, J Biol Chem, 273, 16434-16441). Furthermore, SkiP has been identified as novel binding partner of E7, the major transforming protein of human papillomavirus (Prathapam et al., 2001, Oncogene, 20, 7677-7685). This interaction leads to inhibition of the transcriptional activation activity of SkiP and may promote the transforming activities of E7. Findings from this study demonstrate over-expression of SKIP in OVCA cell lines and abrogation of anchorage-independent growth in these OVCA cells when SKIP expression was abrogated by an antisense ODN. These findings are consistent with the notion that SKIIP possesses oncogenic properties in OCa cells.

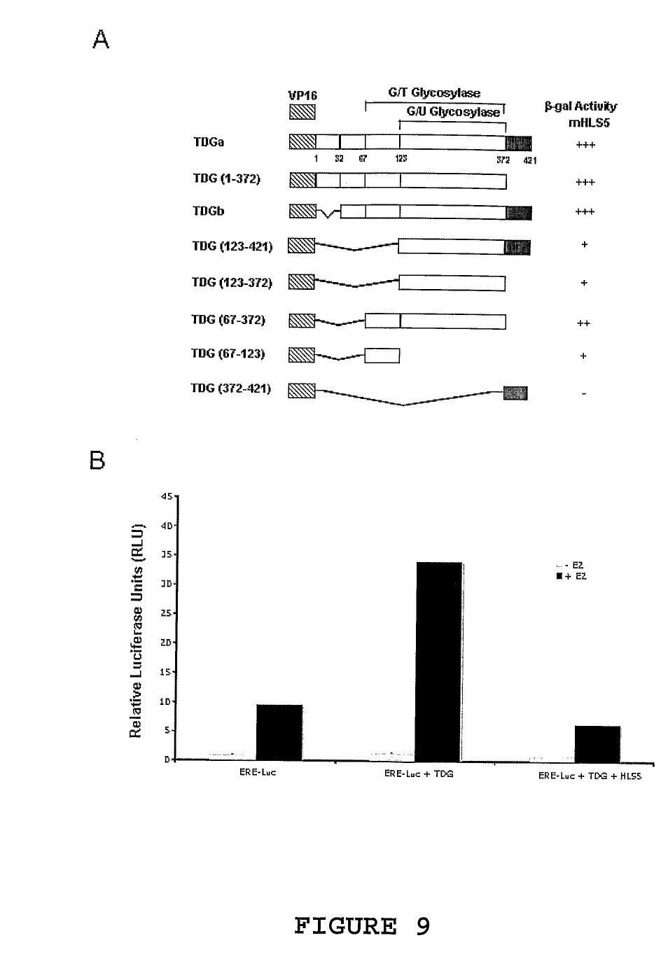

FIG. 9 shows the identification of minimal receptor interaction domains between TDG and in HLS5. Panel (A) shows a schematic representation of Thymine DNA Glycosylase domain structure (sequence numbering corresponds to full-length Murine TDG). The interaction of the different VP16-TDG fusion constructs with HLS5 was evaluated using the Yeast two-hybrid System. The G/T Glycosylase, G/U Glycosylase were found to be essential for this interaction. This same domain was critical for TDG interaction with RAR and RXR (Um, JBC 273:20728, 1998), ERα (Chen, JBC 278:38586, 2003), with CBP/p300 (Tini, Mol Cell 9:265, 2002). Panel (B) shows that HLS5 expression repressed the TDG induced transactivation on the oestrogen response element ERE. The HLS5 expression vector pcDNA3-HLS5 (0.5 μg) and reporter vector ERE-Luciferase (1 μg) were co-transfected into MCF-7 cells in 6-cm dishes, and the effect of pSG5-TDG (0.5 μg)+/−HLS5 on ER transactivation was tested in combination with oestradiol (E2). Cells were also incubated in the absence or presence of 0.1 nM E2. DNA was balanced with equimolar amounts of pcDNA3 empty vector or pcDNA3-HLS5. Vector alone did not inhibit ER transactivation.

Thus, HLS-5 interacts with several molecules which are involved in the regulation of steroid hormone receptor activity.

1. A nuclear receptor modulating agent comprising a compound or composition capable of altering the endogenous levels of HLS-5 or its activity.

2. A method of modulating a nuclear receptor in vivo comprising the step of administering to a subject in need thereof:
(i) an effective amount of a HLS-5 polypeptide, isoform thereof, functional fragment thereof, or composition thereof; or
(ii) a substance capable of altering the endogenous level and/or activity of HLS-5 or an isoform or functional modification or functional fragment thereof; or
(iii) an antibody or fragment thereof which specifically binds HLS-5 or an isoform or functional modification or functional fragment thereof; or
(iv) a polynucleotide molecule which is antisense to a polynucleotide molecule which encodes HLS-5 or an isoform or functional modification or functional fragment thereof; or
(v) combinations thereof.

3. The method of claim 2, wherein the nuclear receptor modulating up-regulates activity of the nuclear receptor.

4. The method of claim 2, wherein the nuclear receptor modulating down-regulates activity of the nuclear receptor.

5. The method of claim 2, wherein the modulating activity is direct.

6. The method of claim 2, wherein the modulating activity is indirect.

7. A method of modulating a nuclear receptor in vitro comprising the step of administering to cells:
(i) an effective amount of a HLS-5 polypeptide, isoform thereof, functional fragment thereof, or composition thereof; or
(ii) a substance capable of altering the endogenous level and/or activity of HLS-5 or an isoform or functional modification or functional fragment thereof; or
(iii) an antibody or fragment thereof which specifically binds HLS-5 or an isoform or functional modification or functional fragment thereof; or
(iv) a polynucleotide molecule which is antisense to a polynucleotide molecule which encodes HLS-5 or an isoform or functional modification or functional fragment thereof; or
(v) combinations thereof.

8. (canceled)

9. A method for treating or preventing a hormone-dependent disorder comprising the step of administering to a subject in need thereof:
(i) a pharmaceutically-effective amount of a HLS-5 polypeptide, isoform thereof, functional fragment thereof, or pharmaceutical composition thereof; or
(ii) a substance capable of altering the endogenous level and/or activity of HLS-5 or an isoform or functional modification or functional fragment thereof; or
(iii) an antibody or fragment thereof which specifically binds HLS-5 or an isoform or functional modification or functional fragment thereof; or
(iv) a polynucleotide molecule which is antisense to a polynucleotide molecule which encodes HLS-5 or an isoform or functional modification or functional fragment thereof; or
(v) combinations thereof.

10. The method of claim 9, wherein the subject is a human, a domestic companion, or zoo animal.

11. The method of claim 9, wherein the companion animal is a dog or a cat.

12. An assay for identifying a modifier of HLS-5 nuclear receptor modulating activity comprising:
(i) providing a nuclear receptor test system including a responsive element capable of binding HLS-5 or fragment thereof, under conditions which permit the binding of HLS-5 or fragment thereof to the responsive element;
(ii) contacting the nuclear receptor test system with HLS-5;
(iii) measuring the level of interaction in the presence of the HLS-5;
(iv) contacting the nuclear receptor system with a candidate modifier of HLS-5 nuclear receptor modulating activity;
(v) measuring the level of interaction in the presence of the candidate modifier; and
(iv) comparing the measured level of interaction in the presence of the candidate modifier with the measured level of interaction in the presence of HLS-5 and/or absence of the candidate modifier, wherein a statistically significant alteration in the interaction in the presence of the candidate modifier is indicative of a modifier of HLS-5 nuclear receptor modulating activity.

13. A method of determining the suitability of HLS-5 treatment of a hormone dependent disorder comprising:
(i) providing a biological sample from a test subject;
(ii) measuring the level of nuclear receptor activity in the biological sample; and
(iii) contacting the biological sample with HLS-5 or functional fragment thereof and measuring the level of nuclear receptor activity and comparing the measured level of nuclear receptor activity in the presence of the HLS-5 with the nuclear receptor activity in the absence of HLS-5, wherein a statistically significant difference in nuclear receptor activity in the presence of the HLS-5 is indicative of the suitability of HLS-5 treatment of the condition.
